(19) United States
(12) Patent Application Publication
(10) Pub. No.: US 2007/0020662 A1
(43) Pub. Date: Jan. 25, 2007

(54) COMPUTERIZED CONTROL OF HIGH-THROUGHPUT EXPERIMENTAL PROCESSING AND DIGITAL ANALYSIS OF COMPARATIVE SAMPLES FOR A COMPOUND OF INTEREST

(75) Inventors: Michael J. Cima, Winchester, MA (US); Douglas A. Levinson, Sherborn, MA (US); Donovan Chin, Lexington, MA (US); Christopher McNulty, Arlington, MA (US); Christopher B. Moore, Cambridge, MA (US); Anthony V. Lemmo, Sudbury, MA (US); Javier P. Gonzalez-Zugasti, N. Billerica, MA (US); Alasdair Y. Johnson, Newburyport, MA (US); Hongming Chen, Acton, MA (US); Orn Almarsson, Shrewsbury, MA (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Transform Pharmaceuticals, Inc., Lexington, MA (US)

(21) Appl. No.: 11/467,061

(22) Filed: Aug. 24, 2006

Related U.S. Application Data
(63) Continuation-in-part of application No. 11/447,592, filed on Jun. 6, 2006.
Application No. 11/447,592 is a continuation of application No. 11/051,517, filed on Jan. 31, 2005, now Pat. No. 7,061,605.
Application No. 11/051,517 is a continuation of application No. 10/235,922, filed on Sep. 6, 2002, now Pat. No. 6,977,723.
Application No. 11/051,517 is a continuation-in-part of application No. 10/142,812, filed on May 10, 2002.
Application No. 11/051,517 is a continuation-in-part of application No. 10/103,983, filed on Mar. 22, 2002.
Application No. 11/051,517 is a continuation-in-part of application No. 09/756,092, filed on Jan. 8, 2001, now abandoned.
Application No. 11/051,517 is a continuation-in-part of application No. 09/628,667, filed on Jul. 28, 2000.
Application No. 09/628,667 is a continuation-in-part of application No. 09/540,462, filed on Mar. 31, 2000, now abandoned.
Application No. 10/103,983 is a continuation-in-part of application No. 09/994,585, filed on Nov. 27, 2001, now Pat. No. 7,108,970.
(60) Provisional application No. 60/318,152, filed on Sep. 7, 2001.
(60) Provisional application No. 60/318,138, filed on Sep. 7, 2001.
(60) Provisional application No. 60/318,157, filed on Sep. 7, 2001.
(60) Provisional application No. 60/290,320, filed on May 11, 2001.
(60) Provisional application No. 60/278,401, filed on Mar. 23, 2001.
(60) Provisional application No. 60/175,047, filed on Jan. 7, 2000.
(60) Provisional application No. 60/196,821, filed on Apr. 13, 2000.
(60) Provisional application No. 60/221,539, filed on Jul. 28, 2000.
(60) Provisional application No. 60/127,755, filed on Apr. 5, 1999.
(60) Provisional application No. 60/253,629, filed on Nov. 28, 2000.

(30) Foreign Application Priority Data
May 18, 2001 (JP) P. 2001-150012
Mar. 25, 2002 (JP) P. 2002-082865

(51) Int. Cl. C12Q 1/68; G01N 33/53; G06F 19/00

(52) U.S. Cl. 435/6; 435/7.1; 702/19; 702/20

(57) ABSTRACT

The present invention relates to computer-controlled automated high-throughput systems and/or computer-program products to design, prepare, process, and analyze a large number of samples having experimental formulations each containing a compound of interest formulated with differing component combinations and varying concentrations and component identities. The computer-controlled methods of the present invention allow determination of the effects of additional or inactive components, such as excipients, carriers, enhancers, adhesives, additives, and the like, on the compound of interest, such as pharmaceuticals. The invention thus encompasses the computer systems, computer methods, and computer-program products for computer-controlled automated high-throughput testing of pharmaceutical compositions or formulations in order to determine the overall optimal composition or formulation for an intended use or purpose.

### CROSS-REFERENCE TO RELATED APPLICATIONS

This application is a continuation-in-part of U.S. patent application Ser. No. 11/447,592, filed Jun. 6, 2006, which is a continuation of U.S. patent application Ser. No. 11/051,517, filed Jan. 31, 2005, now U.S. Pat. No. 7,061,605, which is a continuation of U.S. patent application Ser. No. 10/235,922, filed Sep. 9, 2002, now U.S. Pat. No. 6,977,723 (which claims the benefit of U.S. Provisional Patent Applications Nos. 60/318,152, 60/318,157, and 60/318,138, each of which was filed on Sep. 7, 2001), which is a continuation-in-part of U.S. patent application Ser. No. 10/142,812, filed Jun. 10, 2002 (which claims the benefit of U.S. Provisional Application No. 60/290,320, filed Jun. 11, 2001), which is a continuation-in-part of U.S. patent application Ser. No. 10/103,983, filed Mar. 22, 2002 (which claims the benefit of U.S. Provisional Application No. 60/278,401, filed Mar. 23, 2001), which is a continuation-in-part of U.S. patent application Ser. No. 09/756,092, filed Jan. 8, 2001 (which claims the benefit of U.S. Provisional Application No. 60/175,047, filed Jan. 7, 2000, U.S. Provisional Application No. 60/196,821, filed Apr. 13, 2000, and U.S. Provisional Application No. 60/221,539, filed Jul. 28, 2000), which is a continuation-in-part of U.S. patent application Ser. No. 09/628,667, filed Jul. 28, 2000, which is a continuation-in-part of U.S. patent application Ser. No. 09/540,462, filed Mar. 31, 2000 (which claims the benefit of U.S. Provisional Application No. 60/121,755, filed Apr. 5, 1999), and U.S. patent application Ser. No. 10/103,983 is also a continuation-in-part of U.S. patent application Ser. No. 09/994,585, filed Nov. 27, 2001 (which claims the benefit of U.S. Provisional Application No. 60/253,629, filed Nov. 28, 2000). All the foregoing patents and applications are incorporated herein by reference.

### BACKGROUND OF THE INVENTION

1. The Field of the Invention

The present invention relates to computer-controlled automated high-throughput devices, systems, and methods for conducting and evaluating multiple experiments on samples having different formulations and/or chemical compositions. More particularly, the present invention relates to computer systems, computer methods, and computer-program products for designing, preparing, processing, screening, and analyzing high-throughput preparation and screening of a variety of compounds and compositions in computer-designed arrays.

2. The Related Technology

In recent years, chemical discovery has seen an explosion of new science, such as genomics, proteomic and bioinformatics, as well as the development of high-throughput technologies for identifying and/or creating new compounds or chemical entities. Such technologies allow the researcher to rapidly synthesize and/or identify large numbers of compounds. High-throughput technologies have provided systems that can allow for a large number of compounds to be prepared, and for minor differences in substituents to be varied across the compounds. The compounds can then be tested to determine use for a particular purpose.

Additionally, high-throughput screening technologies have been developed to screen a large number of potentially active compounds against a specific target or for a specific use. The high-throughput screening technologies can utilize array technologies where an array of samples is prepared to include a specific target, such as a biologically active receptor. The large number of potentially active compounds can be tested against the specific target by adding a unique active compound or combination of active compounds to each sample and determining whether or not the compound has activity associated with the specific target. Usually, the samples in an array are substantially similar except for a unique active compound or combination of active compounds. This allows a large number of potentially active compounds to be screened against a specific target without having too many extraneous variables that may affect the screening.

After a compound is found to have sufficient biological activity toward a specific target, the compound is formulated with additional components. Usually, the compound is prepared in a limited number of compositions in order to find a formulation that provides sufficient biological activity for a specific use, such as a specific route of administration. This can include preparing formulations for oral, transdermal, intravenous, and other routes of administration. Often pre-formulated compositions are combined with the active compound to obtain a suitable formulation.

In pharmaceuticals, for example, there are typically trade-offs between drug solubility, stability, absorption and bioavailability. Some active compounds suffer from very low solubility or insolubility in water and undergo extensive first hepatic pass metabolism. Some active compounds suffer from poor absorption due to their low water solubility. While these factors may be taken into consideration during formulation, the experimentation on suitable formulations does not include high-throughput processes similar to those used to identify the active compound. Thus, after large-scale experiments are conducted to find active compounds, the identified compounds are randomly mixed into compositions. Often, the formulation is not analyzed to determine whether or not it is optimal for the intended use.

The solubility, bioavailability, shelf-life, usability, taste and many other properties of the active component may vary in a complex way within the formulation due to interactions among the active component and any additional components. Similarly, properties of a solid form of an active component, such as its crystal habit and morphology, can significantly affect its properties. As such, selection of a formulation for an active component can therefore significantly alter the performance of pharmaceuticals and other chemical products. Dietary supplements, alternative medicines, nutraceuticals, sensory compounds, agrochemicals, and consumer and industrial formulations, can be similarly formulated with formulation issues complicating discovery of a suitable formulation.

The task of determining an optimal or near-optimal formulation is enormous. On the one hand, a property of a formulation often can be optimized only at the expense of other desirable properties, so that no single property may be optimized in isolation. On the other, the properties of an active compound or mixture can vary within formulation parameters in complex or unpredictable ways. Also, the types and ranges of formulation parameters that may be varied in manufacturing are very large.

For example, more than 3,000 excipients are currently accepted and available for designing pharmaceutical compositions. A search for an optimum combination of excipients and active component for even a relatively simple pharmaceutical composition is not trivial. Not only does one need to determine which of those excipients would be compatible with the active agent, but must also to determine the optimum values for such parameters as pH and relative concentrations of the components.

The problem grows geometrically with the number of other components that can be used in formulations and by other parameters that are considered. For example, simply to select a combination of two compounds out of a group of three hundred, without considering other variables such as relative concentrations, requires sifting through 44,850 combinations. This increases rapidly to 4,455,100 combinations for three compounds, and 330,791,175 combinations in the case of a four-compound mixture. Similar problems confront an effort to develop new solid forms of known substances.

In addition, because the conditions under which a formulation is manufactured, stored, administered, or used typically vary over a significant range, the commercial usefulness of a formulation depends on the properties of the formulation over the expected range of conditions under which it will be manufactured, stored, administered or used. If the properties of the formulation change significantly over the expected range, the usefulness of the formulation suffers. Selection of a commercially useful formulation therefore benefits from consideration of the behavior of the formulation or solid form over the expected range.

The magnitude of the problem in finding an optimal formulation does not arise solely from the extremely large number of possible combinations of relevant parameters that may be varied in manufacturing or experimentation. In many situations, neither the experimentally variable parameters nor the measurable or calculable characteristics of an active compound or mixture of interest will have any known correlation with the property or properties which the experimentalist seeks to optimize. In the past, attempts have been made to characterize a material by performing one experiment at a time using a pre-selected combination of additional components and/or one or more bulk properties. This method of characterization is a very time-consuming and ineffective means of finding an optimal formulation. Thus, only a relatively small number of the many possible combinations of chemical entities can be examined.

Therefore, there remains a need in the art for a method for designing, preparing, and screening a large number samples to identify optimal compositions or formulations for an intended use of an active compound. Accordingly, it would be beneficial to have computer-controlled automated systems for high-throughput processing, screening, and analyzing of a large number of samples having different experimental formulations. Additionally, it would be beneficial to have computer systems, computer methods, and computer-program products for designing, preparing, processing, screening, and analyzing formulations of active compounds in computer-designed arrays.

### SUMMARY OF THE INVENTION

The present invention relates to computer-controlled automated high-throughput systems and methods to design, prepare, process, screen, and analyze a large number of samples having experimental formulations, each containing a compound of interest formulated with differing component combinations and/or varying concentrations. The computer-controlled methods of the present invention allow for a determination of the effects of additional or inactive components, such as excipients, carriers, enhancers, adhesives, additives, and the like, on the compound of interest, such as a pharmaceutical. The invention thus encompasses the computer systems, computer methods, and computer-program products for computer-controlled automated high-throughput testing of experimental formulations in order to determine the overall optimal composition or formulation for an intended use or purpose.

In one embodiment, the present invention can include a computing system designed for controlling automated high-throughput processing of an array having a large number of samples in order to identify at least one optimal formulation for a given use of a compound of interest. The computer system can implement a method of computer-aided design for determining an experimental formulation for each sample. Each experimental formulation can have the compound of interest, and the formulations can be based on at least one experimental variable which is varied as to at least some samples. In this way, the effect in terms of changes in the chemical and/or physical properties of the compound of interest due to at least one experimental variable can be identified across a large number of comparative samples.

The computing system can be used in implementing a method of generating and analyzing data from the large number of comparative samples. Such a method can include the following: (a) inputting into the computing system at least one compound of interest to be included in each of a plurality of experimental formulations that are to be designed for the array of samples; (b) inputting into the computer system additional components to be formulated with the at least one compound of interest in the experimental formulations; (c) inputting into the computing system at least one experimental variable to be varied as between at least some of the samples of the array; and (d) the computing system thereafter designing a plurality of unique experimental formulations that differ as between at least some samples of the array based on at least one experimental variable that is varied as between at least some of the samples of the array. Each experimental formulation can be designed at least in part based on at least one experimental variable.

Additionally, the computing system can be used in implementing a method of generating and analyzing data to compare a first group of samples with a second group of samples in the array. Such a method can include the following: (a) inputting into the computing system a compound of interest to be included in each of a plurality of experimental formulations that are to be designed for the array of samples; (b) inputting into the computer system a plurality of additional components to be formulated with the compound of interest in the experimental formulations; (c) inputting into the computing system a plurality of experimental variables to be varied as between at least some of the samples of the array as to at least one of concentration of the compound of interest, concentration of components in the experimental formulations, identity of components, combination of components, additive, solvent, antisolvent composition, temperature, temperature change, heating, cooling, nucleation seeds, supersaturation, pH, pH change, or time of crystallization reaction; (d) the computing system thereafter designing, for a first group of samples in the array, a first plurality of experimental formulations that are different as between at least some of the samples in the first group that are based on a first experimental variable that is varied among the first plurality of experimental formulations determined for the first group; and (e) the computing system also designing, for at least a second group of samples in the array, a second plurality of experimental formulations that are different as between at least some of the samples in the second group that are based on a second experimental variable that is varied as among the second plurality of experimental formulations determined for the second group.

In one embodiment, the computing system can be used to provide a method of computer-aided design and processing of an experimental formulation for each sample. Such a method can include the following: (a) inputting into the computing system at least one compound of interest and any additional components to be included in a plurality of experimental formulations that are to be designed for the array of samples; (b) inputting into the computing system at least one selected experimental variable of interest that is to be varied as between at least some of the samples of the array; (c) the computing system thereafter designing a plurality of unique experimental formulations that differ as between at least some samples of the array based on the at least one selected experimental variable of interest that is varied as between the at least some samples of the array; (d) the computing system thereafter controlling a process by which an experimental formulation for each sample is prepared and tested in order to create changes across a large number of comparative samples for the at least one compound of interest in its chemical and/or physical properties; (e) inputting into the computing system detected changes across the large number of comparative samples for the at least one compound of interest; and (f) the computing system thereafter automatically screening the large number of samples by identifying those samples which contain chemical and/or physical properties likely to lead to an optimal formulation for a given use of a compound of interest.

In one embodiment, the computing system can be used to provide a method of directed computer-aided design and processing of an experimental formulation for each sample in a first array which then uses data obtained from the first array to design and process an experimental formulation for each sample in a second array. Often the first array will include samples that contain different additional components, while the second array will differ as to concentration of the components. Such a method can include the following: (a) inputting into the computing system at least one compound of interest and any additional components to be included in a plurality of experimental formulations that are to be designed for a first array of samples; (b) inputting into the computing system at least one selected experimental variable of interest that is to be varied as between at least some samples of the first array; (c) the computing system thereafter designing a plurality of unique experimental formulations that differ as between at least some samples based on the at least one selected experimental variable of interest that is varied as between the at least some samples of the first array; (d) the computing system thereafter controlling a process by which an experimental formulation for each sample is prepared and tested in order to create changes in chemical and/or physical properties across a large number of comparative samples for the at least one compound of interest; (e) inputting into the computing system detected changes across the large number of comparative samples for the at least one compound of interest; (f) the computing system thereafter screening the large number of samples by identifying those samples which contain chemical and/or physical properties likely to lead to an optimal formulation for a given use of a compound of interest, and storing as a first data set information as to the experimental formulation and the resulting chemical and/or physical properties for each of the identified samples; (g) inputting to the computing system at least one other selected experimental variable of interest that is to be varied as between at least some identified samples of the first data set; (h) the computing system thereafter designing a plurality of further experimental formulations for a second array having a large number of samples that are different as between at least some of the identified samples of the first data set based on the at least one further selected experimental variable of interest that is to be varied as between the at least some identified samples of the first data set; (i) the computing system thereafter controlling a process by which the plurality of further experimental formulations in the second array of samples are prepared and tested in order to create further changes in chemical and/or physical properties across further comparative samples for the at least one compound of interest; (j) inputting into the computing system detected further changes across the further comparative samples of the first data set for the at least one compound of interest; (k) the computing system thereafter screening the further comparative samples by identifying changes in chemical and/or physical properties and storing as a second data set information as to the plurality of further experimental formulations and the resulting chemical and/or physical properties for each further comparative sample; and (l) the computing system thereafter selecting from the first and second data sets those samples which contain chemical and/or physical properties likely to lead to an optimal formulation for a given use of a compound of interest.

In one embodiment, the present invention can include a computer-program product to operate with a computing system designed for controlling automated high-throughput processing of an array having a large number of samples in order to identify chemical and/or physical properties leading to optimal formulation for a given use of a compound of interest. The computer-program product can be used for implementing a method of computer-aided design for determining an experimental formulation for each sample. Each experimental formulation can be designed based on at least one experimental variable which is varied as to at least some samples so that the effect in terms of changes in the chemical and/or physical properties of the compound of interest due to at least one experimental variable can be identified across a large number of comparative samples. The computer-program product can include a computer-readable medium, which are well-known in the art, containing computer-executable instructions for causing the computing system to execute the method.

The computer-program product can be used in implementing a method of generating and analyzing data from the large number of comparative samples. Such a method can include the following: (a) inputting into the computing system at least one compound of interest and any additional components to be included in each of a plurality of experimental formulations that are to be designed for the array of samples; (b) inputting into the computer system additional components to be formulated with the at least one compound of interest in the experimental formulations; (c) inputting into the computing system at least one experimental variable to be varied as between at least some of the samples of the array; and (d) the computing system thereafter designing a plurality of unique experimental formulations that differ as between at least some samples based on at least one experimental variable that is varied as between the at least some samples of the array, each experimental formulation being designed at least in part based on the at least one experimental variable.

Additionally, the computer-program product can be used in implementing a method of generating and analyzing data to compare a first group of samples with a second group of samples in the array. Such a method can include the following: (a) inputting into the computing system a compound of interest to be included in each of a plurality of experimental formulations that are to be designed for the array of samples; (b) inputting into the computer system a plurality of additional components to be formulated with the compound of interest in the experimental formulations; (c) inputting into the computing system a plurality of experimental variables to be varied as between at least some of the samples of the array as to at least one of concentration of the compound of interest, concentration of components in the experimental formulations, identity of components, combination of components, additive, solvent, antisolvent composition, temperature, temperature change, heating, cooling, nucleation seeds, supersaturation, pH, pH change, or time of crystallization reaction; (d) the computing system thereafter designing, for a first group of samples in the array a first plurality of experimental formulations that are different as between at least some of the samples in the first group that are based on a first experimental variable that is varied among the first plurality of experimental formulations determined for the first group; and (e) the computing system also designing, for at least a second group of samples in the array a second plurality of experimental formulations that are different as between at least some of the samples in the second group that are based on a second experimental variable that is varied as among the second plurality of experimental formulations determined for the second group.

In one embodiment, the computer-program product can be used in implementing a method computer-aided design and processing of an experimental formulation for each sample. Such a method can include the following: (a) inputting into the computing system at least one compound of interest and any additional components to be included in a plurality of experimental formulations that are to be designed for the array of samples; (b) inputting into the computing system at least one selected experimental variable of interest that is to be varied as between at least some of the samples of the array; (c) the computing system thereafter designing a plurality of unique experimental formulations that differ as between at least some samples of the array based on the at least one selected experimental variable of interest that is varied as between the at least some samples of the array; (d) the computing system thereafter controlling a process by which an experimental formulation for each sample is prepared and tested in order to create changes across a large number of comparative samples for the at least one compound of interest in its chemical and/or physical properties; (e) inputting into the computing system detected changes across the large number of comparative samples for the at least one compound of interest; and (f) the computing system thereafter automatically screening the large number of samples by identifying those samples which contain chemical and/or physical properties likely to lead to an optimal formulation for a given use of a compound of interest.

In one embodiment, the computer-program product can be used to provide a method of directed computer-aided design and processing of an experimental formulation for each sample in a first array and using data obtained from the first array to design and process an experimental formulation for each sample in a second array. Often the first array will include samples that differ in identity of the additional components and the second array will differ in the concentration of the additional components identified from the first array. Such a method can include the following: (a) inputting into the computing system at least one compound of interest and any additional components to be included in each of a plurality of experimental formulations that are to be designed for the array of samples; (b) inputting into the computing system at least one selected experimental variable of interest that is to be varied as between at least some samples of the array; (c) the computing system thereafter designing a plurality of unique experimental formulations that differ as between at least some samples based on the at least one selected experimental variable of interest that is varied as between the at least some samples of the array; (d) the computing system thereafter controlling a process by which an experimental formulation for each sample is tested in order to create changes in chemical and/or physical properties across a large number of comparative samples for the at least one compound of interest; (e) inputting into the computing system detected changes across the large number of comparative samples for the at least one compound of interest; (f) the computing system thereafter screening the large number of samples by identifying those samples which contain chemical and/or physical properties likely to lead to an optimal formulation for a given use of a compound of interest, and storing as a first data set information as to the experimental formulation and the resulting chemical and/or physical properties for each of the identified samples; (g) inputting to the computing system at least one other selected experimental variable of interest that is to be varied as between at least some identified samples of the first data set; (h) the computing system thereafter designing a plurality of further experimental formulations for a second array having a large number of samples that are different as between at least some of the identified samples of the first data set based on the at least one further selected experimental variable of interest that is to be varied as between the at least some identified samples of the first data set; (i) the computing system thereafter controlling a process by which the plurality of further experimental formulations in the second array of samples are prepared and tested in order to create further changes in chemical and/or physical properties across further comparative samples for the at least one compound of interest; (j) inputting into the computing system detected further changes across the further comparative samples of the first data set for the at least one compound of interest; (k) the computing system thereafter screening the further comparative samples of the first data set by identifying changes in chemical and/or physical properties and storing as a second data set information as to the plurality of further experimental formulations and the resulting chemical and/or physical properties for each further comparative sample; and (l) the computing system thereafter selecting from the first and second data sets those samples which contain chemical and/or physical properties likely to lead to an optimal formulation for a given use of a compound of interest.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings, in which:

FIG. 1 is a schematic diagram illustrating an embodiment of a high-throughput process for preparing arrays of samples containing an embodiment of a compound of interest and analyzing the individual samples.

FIG. 2A is a schematic diagram illustrating an embodiment of a system for conducting the high-throughput process of FIG. 1.

FIG. 2B is a schematic diagram of an embodiment of a sample preparation module for the system of FIG. 2A.

FIG. 2C is a schematic diagram of an embodiment of incubation and scanning modules for the system of FIG. 2A.

FIG. 3 is a schematic diagram illustrating an embodiment of a high-throughput process for a directed search strategy.

FIG. 4 is a schematic diagram illustrating an embodiment of a high-throughput process for a directed search strategy.

FIG. 5 is a schematic diagram illustrating an embodiment of a high-throughput process including models for determining and screening experimental formulations.

FIG. 6 is a schematic diagram illustrating architecture of one embodiment of a computing system for controlling automated high-throughput systems.

FIG. 7 is a schematic diagram illustrating an embodiment of a high-throughput process to assess collection of experimental results in a search for novel or known solid forms.

FIG. 8 is a schematic diagram illustrating an embodiment of a high-throughput process for analyzing data.

### DETAILED DESCRIPTION OF THE PREFERRED EMBODIMENTS

The present invention relates to computer-controlled automated high throughput systems, computer-program products, and computer-controlled methods for processing an array having a large number of samples in order to identify at least one optimal formulation for a given use of a compound of interest. The computer system can implement a method of computer-aided design for determining an experimental formulation and experimental process for each sample. Each experimental formulation can have the compound of interest and the formulations can be based on at least one experimental variable which is varied as to at least some samples so that the effect in terms of changes in the chemical and/or physical properties of the compound of interest due to at least one experimental variable can be identified across a large number of comparative samples for a compound of interest. The computer-controlled systems, computer-program products, and methods of the present invention may be used to design, prepare, process, screen, analyze, and identify the optimal components (e.g., solvents, carriers, transport enhancers, adhesives, additives, and other excipients) for various chemical formulations.

I. Introduction

As an alternate approach to traditional methods for discovery of new or optimal formulations and discovery of conditions relating to formation, inhibition of formation, or dissolution of solid forms, a computer-controlled automated high-throughput system and methods of use can design, produce, and screen hundreds, thousands, to hundreds of thousands of samples per day. The array technology described herein is a computer-controlled high-throughput approach that can be used to generate large numbers (e.g., greater than 10, more typically greater than 50 or 100, and more preferably 1000 or greater samples) of parallel small-scale formulation experiments (e.g., crystallizations) for a given compound of interest.

Typically, each sample is designed and prepared to have less than about 1 g of the compound of interest, preferably, less than about 100 mg, more preferably, less than about 25 mg, even more preferably, less than about 1 mg, still more preferably less than about 100 micrograms, and optimally less than about 100 nanograms of the compound of interest. The computer-controlled systems and methods are useful to optimize, select, and discover new or optimal formulations having enhanced properties. In some instances, the formulations produce novel solid forms of the compound of interest. The computer-controlled systems and methods are also useful to discover compositions or formulation conditions that promote formation of formulations with desirable properties. The computer-controlled systems and methods are further useful to discover compositions or conditions that inhibit, prevent, or reverse formation of specific solid forms within formulations.

The computer-controlled automated high-throughput system and methods can design and prepare an array of sample sites, such as a 24, 48 or 96-well plate or more samples. Each sample in the array can include a mixture of a compound of interest and at least one other additional component. The array of samples can be subjected to a set of processing parameters designed and implemented by the computing system. Examples of processing parameters that can be varied to form different formulations can include adjusting the temperature; adjusting the time; adjusting the pH; adjusting the amount or the concentration of the compound of interest; adjusting the amount or the concentration of a component; component identity (adding one or more additional components); adjusting the solvent removal rate; introducing of a nucleation event; introducing of a precipitation event; controlling evaporation of the solvent (e.g., adjusting a value of pressure or adjusting the evaporative surface area); and adjusting the solvent composition.

The contents of each sample in the processed array are typically analyzed initially for physical or structural properties; for example, the likelihood of crystal formation is assessed by turbidity, using a device such as a spectrophotometer. However, a simple visual analysis can also be conducted including photographic analysis. For example, the formulation can be analyzed in order to detect a solid, crystalline, or amorphous form of the compound of interest. Also, more specific properties of the solid can then be measured, such as polymorphic form, crystal habit, particle size distribution, surface-to-volume ratio, and chemical and physical stability, and the like. Samples containing active compounds can be screened to analyze properties of the formulation, such as altered bioavailability and pharmacokinetics. The active compounds can be screened in vitro for their pharmacokinetics, such as absorption through the gut (for an oral preparation), skin (for transdermal application), or mucosa (for nasal, buccal, vaginal or rectal preparations), solubility, degradation or clearance by uptake into the reticuloendothelial system (“RES”) or excretion through the liver or kidneys following administration, then tested in vivo in animals. Testing of the large number of samples can be done simultaneously or sequentially.

The computer-controlled automated high-throughput system and methods are widely applicable for different types of active compounds (e.g., compound of interest), including pharmaceuticals, dietary supplements, alternative medicines, nutraceuticals, sensory compounds, agrochemicals, the active component of a consumer formulation, and the active component of an industrial formulation. Accordingly, optimal formulations for a variety of active compounds can be determined by using a high-throughput approach with the computer-controlled systems and methods of the present invention.

The computer-controlled systems inherently include computer-program products in order to provide executable instructions to control the computing system and automated equipment operated by the computing systems. As such, methods performed by computer-controlled systems or any automated equipment are inherently controlled by computer-program products, which are usually in the form of software.

A. Definitions

As used herein, the term “array” is meant to refer to a plurality of samples having a plurality of distinct experimental formulations. Preferably, an array includes at least 24 samples each comprising an experimental formulation with a compound of interest and at least one additional component. An array can comprise one or more groups of samples also known as sub-arrays. For example, a group can be a 96-tube plate of sample tubes or a 96-well plate of sample wells in an array consisting of 100 or more plates. Each sample or selected samples or each sample group of selected sample groups in the array can be subjected to the same or different processing parameters. Each sample or sample group can have different components or concentrations of components to induce, inhibit, prevent, or reverse formation of solid forms of the compound of interest. Arrays can be prepared by preparing a plurality of samples, each sample comprising a compound of interest and one or more components, then processing the samples to induce, inhibit, prevent, or reverse formation of solid forms of the compound of interest.

As used herein, the term “automated” or “automatically” is meant to refer to the use of computer software, computer systems, and computer-controlled robotics to design, add, mix, process, screen, and analyze the samples. Computer systems and software media known in the art may be utilized in controlling the inventive systems and implementing the inventive processes.

As used herein, the terms “automated experimentation apparatus,” “computer-controlled automated system,” and “computer-controlled-automated high-throughput system” are meant to refer to a system of experimental equipment that is controlled by a computing system for performing large numbers of experiments having at least one experimental step performed by computer-controlled apparatus. Human operators may direct the apparatus, or manually perform some portions of the process (e.g. moving groups of plates from one automated station to another, or performing an experimental procedure on results identified using a computer). In some instances the computer-controlled systems for performing large numbers of experiments can include all experimental steps being performed by computer-controlled apparatus.

As used herein, the term “component” is meant to refer to any substance that is combined, mixed, or processed with the compound of interest to form a sample. The term component also encompasses the compound of interest itself Components can be large molecules (i.e., molecules having a molecular weight of greater than about 1000 g/mol), such as large-molecule pharmaceuticals, oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol) such as small-molecule pharmaceuticals, hormones, nucleotides, nucleosides, steroids, or amino acids. A component can be a substance whose intended effect in an array sample is to induce, inhibit, prevent, or reverse formation of solid forms of the compound of interest.

As used herein, the term “compound of interest” is meant to refer to the active component present in array samples where the array is designed to study its physical or chemical properties. Preferably, a compound of interest is a particular active compound for which it is desired to identify solid forms or solid forms with enhanced properties. The compound of interest may also be a particular compound for which it is desired to find conditions or compositions that inhibit, prevent, or reverse solidification. Preferably, the compound of interest is present in every sample of the array, with the exception of negative controls. Examples of compounds-of-interest include, but are not limited to, pharmaceuticals, dietary supplements, alternative medicines, nutraceuticals, sensory compounds, agrochemicals, the active component of a consumer formulation, and the active component of an industrial formulation.

As used herein, the term “excipient” is meant to refer to the substances used to formulate an active compound into a pharmaceutical formulation. Preferably, an excipient does not lower or interfere with the primary effect of the active compound. More preferably, an excipient is inert. The term “excipient” encompasses carriers, solvents, diluents, vehicles, stabilizers, and binders. Excipients can also be those substances present in a pharmaceutical formulation as an indirect result of the manufacturing process. Preferably, excipients are approved for or considered to be safe for human and animal administration.

As used herein, the term “experimental parameters” is meant to refer to the physical or chemical conditions under which a sample is subjected and the time during which the sample is subjected to such conditions. Experimental parameters include, but are not limited to, temperature, time, pH, amount or concentration of a component, component identity, solvent removal rate, and solvent composition. Sub-arrays or even individual samples within an array can be subjected to processing parameters that are different from the processing parameters to which other sub-arrays or samples within the same array are subjected. Processing parameters will differ between sub-arrays or samples when they are intentionally varied to induce a measurable change in the properties of the sample.

As used herein, the term “model” is meant to refer to a computational entity that accepts as inputs data representing values of experimental parameters and/or results and produces as output data representing an estimate of one or more properties expected to result from an experiment corresponding to the input.

As used herein, the term “pharmaceutical” is meant to refer to any substance that has a therapeutic, disease preventive, diagnostic, or prophylactic effect when administered to an animal or a human. The term pharmaceutical includes prescription pharmaceuticals and over the counter pharmaceuticals. Pharmaceuticals suitable for use in the invention include all those known or to be developed. A pharmaceutical can be a large or small molecule as defined hereinabove.

As used herein, the term “physical state” of a component or a compound of interest is initially defined by whether the component is a liquid, a solid, or the like. If the component is a solid, the physical state is further defined by the particle or crystal size and particle-size distribution.

As used herein, the term “property” is meant to refer to a structural, physical, pharmacological, or chemical characteristic of a sample; preferably, a structural, physical, pharmacological, or chemical characteristic of a compound of interest. Structural properties include, but are not limited to, whether the compound of interest is crystalline or amorphous, and if crystalline, the polymorphic form and a description of the crystal habit. Structural properties also include the composition, such as whether the solid form is a hydrate, solvate, or a salt. Preferred properties are those that relate to the efficacy, safety, stability, or utility of the compound of interest, such as stability, solubility, dissolution, permeability, and partitioning; mechanical properties, such as compressibility, compactability, and flow characteristics; the sensory properties of the formulation, such as color, taste, and smell; and properties that affect the utility, such as absorption, bioavailability, toxicity, metabolic profile, and potency.

A physical property can include, but is not limited to, physical stability, melting point, solubility, strength, hardness, compressibility, and compactability. Physical stability refers to the ability of a compound or composition to maintain its physical form, for example, maintenance of particle size, crystal or amorphous form, complexed form (such as hydrates and solvates), and mechanical properties, such as compressibility and flow characteristics, and resistance to absorption of ambient moisture. Methods for measuring physical stability include spectroscopy, sieving or testing, microscopy, sedimentation, stream scanning, and light scattering. Polymorphic changes, for example, are usually detected by differential scanning calorimetry or quantitative infrared analysis.

A chemical property can include, but is not limited to chemical stability, such as susceptibility to oxidation and reactivity with other compounds, such as acids, bases, or chelating agents. Chemical stability refers to resistance to chemical reactions induced, for example, by heat, ultraviolet radiation, moisture, chemical reactions between components, or oxygen. Well known methods for measuring chemical stability include mass spectroscopy, UV-VIS spectroscopy, HPLC, gas chromatography, and liquid chromatography-mass spectroscopy (LC-MS).

As used herein, the term “processing parameters” is meant to refer to the physical or chemical conditions under which a sample is subjected and the time during which the sample is subjected to such conditions. Processing parameters include, but are not limited to, adjusting the temperature; time; pH; amount or concentration of the compound of interest; amount or concentration of a component; component identity (adding one or more additional components); adjusting the solvent removal rate; introduction of a nucleation or precipitation event; controlling evaporation of the solvent (e.g., adjusting a value of pressure or adjusting the evaporative surface area); and adjusting the solvent composition.

As used herein, the term “sample” is meant to refer to a mixture of a compound of interest and one or more additional components to be subjected to various processing parameters and then screened to detect the presence or absence of solid forms, preferably, to detect desired solid forms with new or enhanced properties. In addition to the compound of interest, the sample can comprise one or more components; preferably, 2 or more components; more preferably, 3 or more components. In general, a sample will comprise one compound of interest, but can comprise multiple compounds-of-interest. Typically, a sample comprises less than about 1 g of the compound of interest; preferably, less than about 100 mg; more preferably, less than about 25 mg; even more preferably, less than about 1 mg; still more preferably, less than about 100 micrograms; and optimally, less than about 100 nanograms of the compound of interest. Preferably, the sample has a total volume of 100-250 μL. A sample can be contained in any container or holder, or be present on any substance or surface, or absorbed or adsorbed in any substance or surface. The only requirement is that the samples are isolated from one another, that is, located at separate sites. In one embodiment, samples are contained in sample wells in standard sample plates, for instance, in 24-, 36-, 48-, or 96-well plates or more (or filter plates) of volume 250 μL commercially available, for example, from Millipore, Bedford, Mass.

As used herein, the term “solid form” is meant to refer to a form of a solid substance, element, or chemical compound that is defined and differentiated from other solid forms according to its physical state and properties.

II. Computer-Controlled Automated High-Throughput System

In one embodiment, the present invention is directed, in part, to computer-controlled automated high-throughput systems and/or computer-program products (e.g., software) for determining conditions that when applied to a particular compound or composition provide a particular result (e.g. a compound or composition having particular chemical and/or physical properties). The invention is further directed to computer-controlled systems and methods for the generation, synthesis, and/or identification of various forms of a compound or composition, such as, but not limited to, polymorphs, salts, hydrates, solvates, desolvates, and amorphous forms. The invention is also directed to methods and systems for the generation, synthesis, and/or identification of various forms of solids such as, but not limited to, crystal habit and particle size distribution.

The invention encompasses a computer-controlled system and software for planning (i.e., designing) and conducting high-throughput experiments on one or more arrays of samples. The system encompasses various computer-controlled equipment and software to implement methods that can be used to design, prepare, process, screen, and analyze samples. Additionally, the computer-controlled equipment and software can be used to inspect, process, and screen samples. The computer-controlled equipment and software can be used to collect spectroscopic and other data from one or more of the samples. The computer-controlled equipment and software can be used to process, interpret, and analyze the data. The system can include robotics, computers, spectral techniques, and various mechanical devices, each designed to conduct high-throughput experiments on large or preferably small amounts of material, including materials on the milligram and microgram scales.

A. Sample and Process Design

In one embodiment, the present invention can include a computing system designed for controlling automated high-throughput preparation and processing of an array having a large number of samples. As such, the computing system can implement a method of computer-aided design for determining an experimental formulation and experimental processing for each sample. Each experimental formulation can have the compound of interest, and the formulations can be based on at least one experimental variable which is varied as to at least some samples so that the effect in terms of changes in the chemical and/or physical properties of the compound of interest due to at least one experimental variable can be identified across a large number of comparative samples for a compound of interest. Also, the sample processing can be varied to determine whether or not various processes can effect the chemical and/or physical properties of the compound of interest

The computing system can be used in implementing a method of designing an experimental formulation for each of a large number of comparative samples. Such a method of designing experimental formulations can include inputting into the computing system at least one compound of interest to be included in each of a plurality of experimental formulations that are to be designed for the array of samples. Also, the additional components to be formulated with the at least one compound of interest in the experimental formulations can be input into the computing system. Additionally, least one experimental variable to be varied as between at least some of the samples of the array can be input into the computing system. In part, this can include identifying specific values or ranges of values in varying the variables. Accordingly, the computing system thereafter can design a plurality of unique experimental formulations that differ as between at least some samples of the array based on at least one experimental variable that is varied as between the at least some samples of the array. Each experimental formulation being designed is at least in part based on at least one experimental variable and the compound of interest.

For example, the combinations of the compound of interest and various components at various concentrations and combinations can be generated using standard formulating software (e.g. Matlab software, commercially available from Mathworks, Natick, Mass.). The combinations thus generated can be downloaded into a spread sheet, such as Microsoft EXCEL. From the spread sheet, a work list can be generated for instructing the automated distribution mechanism to prepare an array of samples according to the various combinations generated by the formulating software. The work list can be generated using standard programming methods according to the automated distribution mechanism that is being used. The use of so-called work lists simply allows a file to be used as the process command rather than discrete programmed steps. The work list combines the formulation output of the formulating program with the appropriate commands in a file format directly readable by the automatic distribution mechanism. However, various computer-program products can be used for generating arrays of samples having different experimental formulations, and such computer-program products can be operated on a computer within the computing system.

In one embodiment, the experimental variable to be varied as between at least some samples of the array is varied as to at least one of concentration of the compound of interest, concentration of components in the experimental formulations, identity of the components, combination of components, additive, solvent, antisolvent composition, temperature, temperature change, heating, cooling, nucleation seeds, supersaturation, pH, pH change, or time of crystallization reaction.

In one embodiment, at least one criteria can be input into the computing system for determining the effect of at least one experimental variable for each experimental formulation that is varied as to that experimental variable. The effect of the criteria can be manifested by a change in one or more of the physical property permutations for the compound of interest between different experimental formulations. The effects can be identified by changes in microstructure, crystallinity, amorphism, polymorphism, hydrate, solvate, isomorphic desolvate, packing order, ionic crystal, interstitial space, lattice, or habit.

In one embodiment, the computing system can design a process for processing the array of samples to determine an effect on the compound of interest of at least one experimental variable for each experimental formulation. Such processing can be determined from the experimental variable input into the computing system so as to process the samples as described herein. For example, the processing of each experimental formulation can include a process consisting of at least one of mixing, agitating, heating, cooling, adjusting pressure, adding crystallization aids, adding nucleation promoters, adding nucleation inhibitors, adding acids, adding bases, stirring, milling, filtering, centrifuging, emulsifying, mechanical stimulation, introducing ultrasound or laser energy to the experimental formulation, subjecting the experimental formulation to a temperature gradient, allowing the experimental formulation to set for a time, or heating to a first temperature then cooling to a second temperature.

In one embodiment, the present invention can include the computer-controlled automated high-throughput system implementing a method for using a computer-program product having computer-modeling capabilities for determining at least one optimal formulation of a compound of interest, such as a pharmaceutical, for a desired purpose. In some instances, the formulation can include a solid form of the compound of interest. The computer-controlled system and/or computer-program product can design and screen the compound of interest. The computer-controlled system and/or computer-program product can compute an optimization algorithm in order to select a plurality of molecular descriptors and a model accepting the molecular descriptors as parameters to optimize the design and/or predictive power of the computer-modeling capabilities. The molecular descriptors and model can be used in designing and testing a large number of samples having experimental formulations to determine at least one optimal formulation for the compound of interest.

Additionally, the computer-controlled system and/or computer-program product can generate values of experimental parameters using the model to design experimental formulations and processes for an array of samples. As such, high-throughput design and screening can be performed as described herein by using the values generated by the model. Also, experimental results obtained from screening the experimental formulations designed by the model can be compared with the results predicted by the model. The model and/or experimental parameters used therewith can be modulated based on the high-throughput experimental results.

The model-generated values can be used to find an extremum of an expected property of an experiment, boundaries between solid forms, regions in which desired properties of formulations change rapidly with respect to changes experimental parameters, regions in which desired properties of formulations change slowly with respect to changes experimental parameters, or regions of ambiguity or low confidence in classification or regression results. As such, the predictive power of the model can be determined with respect to an extremum of an expected property of an experiment, with respect to boundaries between solid forms, with respect to regions in which desired properties of formulations or solid forms change rapidly with respect to changes in experimental parameters, or with respect to one or more regions within class boundaries.

Also, a variety of optimization algorithms and models may be used in the computing system and/or computer-program product. Accordingly, an approximately maximally diverse set of values of experimental parameters for high-throughput screening can be generated using a diversification algorithm and a metric for measuring diversification. Alternatively, a set of values for experimental parameters for high-throughput screening can be generated based on a structure-activity model.

B. Sample Preparation

The computer-controlled automated high-throughput system can include an automated distribution mechanism to add components and the compound of interest to separate sites; for example, on an array plate having sample wells or sample tubes. Preferably, the distribution mechanism is controlled by computer software, such as a computer-program product operating on the computing system, and can vary at least one variable with respect to the experimental formulation containing the compound of interest. As such, the distribution mechanism can vary the identity of the component(s), the component concentration, and the like. Also, the distribution mechanism can prepare the sample in accordance with the experimental formulation designed by the computing system. Material handling technologies and robotics can be used in the distribution mechanism and are well known to those skilled in the art. Of course, if desired, individual components can be placed at the appropriate sample site manually. This pick and place technique is also known to those skilled in the art.

Also, the computer-controlled system can include a processing mechanism to process the samples after component addition. Optionally, the processing mechanism can have a processing station to process the samples after preparation. A processing mechanism can be any computer-controlled equipment that can process the array of samples by any of the processes described herein.

Additionally, the computer-controlled system can include a screening mechanism to test each sample to detect a change in physical and/or chemical properties of the formulation and compound of interest. Preferably, the testing mechanism is automated and controlled by computer software, such as a computer-program product operating on the computing system,

A number of companies have developed array systems that can be adapted for use in the invention disclosed herein. Accordingly, array systems can be employed in a computer-controlled system as described herein. Such array systems may require modification, which is well within ordinary skill in the art. Examples of companies having array systems include Gene Logic of Gaithersburg, Md. (see U.S. Pat. No. 5,843,767 to Beattie); Luminex Corp., Austin, Tes.; Beckman Instruments, Fullerton, Calif.; MicroFab Technologies, Plano, Tex.; Nanogen, San Diego, Calif.; and Hyseq, Sunnyvale, Calif. These devices test samples based on a variety of different systems. All include thousands of microscopic channels that direct components into test wells, where reactions can occur. These systems are connected to computers for analysis of the data using appropriate software and data sets. The Beckman Instruments system can deliver nanoliter samples of 96- or 384-arrays, and is particularly well-suited for hybridization analysis of nucleotide molecule sequences. The MicroFab Technologies system delivers sample using inkjet printers to aliquot discrete samples into wells. These and other systems can be adapted as required for use herein.

The automated distribution mechanism can deliver at least one compound of interest, such as a pharmaceutical, as well as various additional components, such as solvents and additives, to each sample well. Preferably, the automated distribution mechanism can deliver multiple amounts of each component. Automated liquid and solid distribution systems are well known and commercially available, such as the Tecan Genesis, from Tecan-US, RTP, North Carolina. The robotic arm can collect and dispense the solutions, solvents, additives, or compound of interest from the stock plate to a sample well or sample tube. The process is repeated until the array is completed, for example, generating an array that moves from wells at left to right and from top to bottom in increasing polarity or non-polarity of solvent. The samples are then mixed. For example, the robotic arm moves up and down in each well plate for a set number of times to ensure proper mixing.

Liquid handling devices manufactured by vendors such as Tecan, Hamilton and Advanced Chemtech are all capable of being used in the invention. The liquid handling device specifically manufactured for organic syntheses are the most desirable for application to crystallization due to the chemical compatibility issues. Robbins Scientific manufactures the Flexchem reaction block which consists of a Teflon reaction block with removable gasketed top and bottom plates. This reaction block is in the standard footprint of a 96-well microtiter plate and provides for individually sealed reaction chambers for each well. The gasketing material is typically Viton, neoprene/Viton, or Teflon-coated Viton, and acts as a septum to seal each well. As a result, the pipetting tips of the liquid handling system need to have septum-piercing capability. The Flexchem reaction vessel is designed to be reusable in that the reaction block can be cleaned and reused with new gasket material.

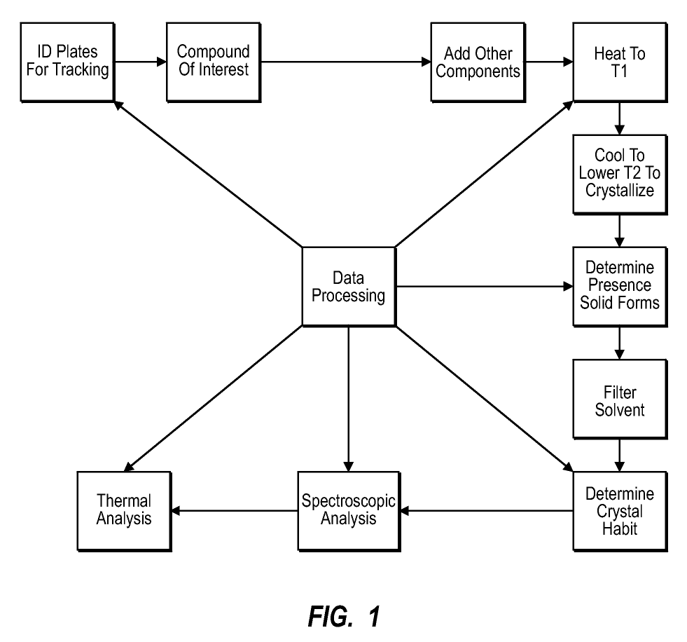

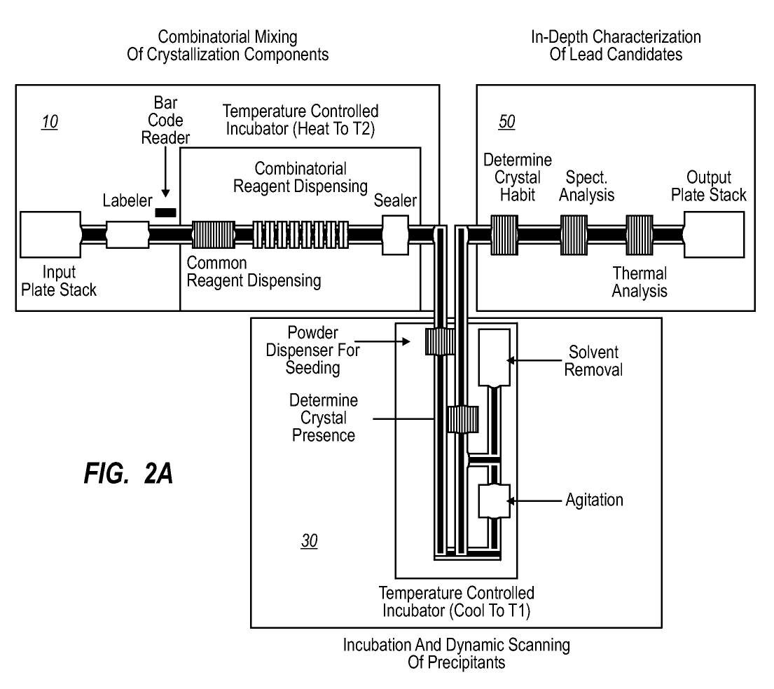

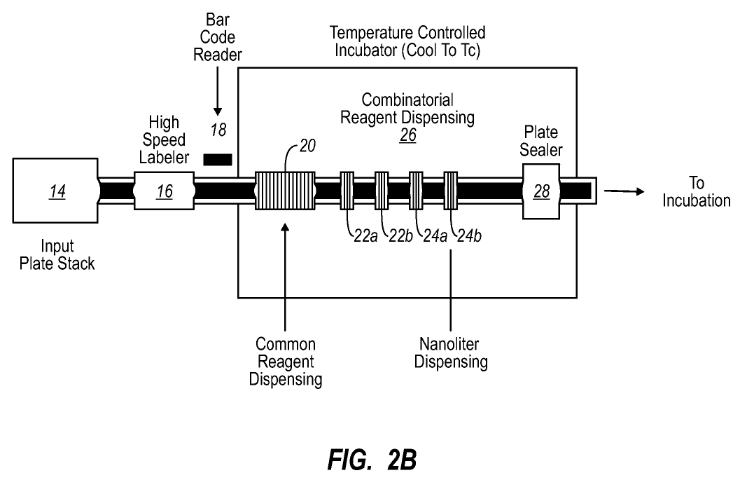

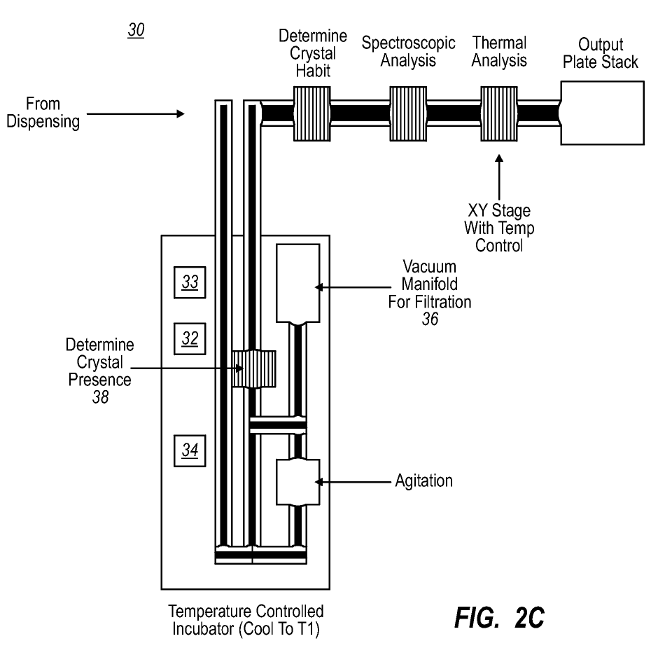

An schematic diagram of an exemplary computer-controlled system and process is shown in FIGS. 1 and 2A-2C. The computer-controlled system consists of a series of integrated modules, or workstations. These modules can be connected directly, through an assembly-line approach, using conveyor belts, or can be indirectly connected by human intervention to move substances between modules. As shown, plates are identified for tracking. Next, the compound of interest is added followed by various other components, such as solvents and additives. Preferably, the compound of interest and all components are added by an automated distribution mechanism. The array of samples is then heated to a temperature (T1), preferably to a temperature at which the active component is completely in solution. The samples are then cooled to a lower temperature (T2) usually for at least one hour. If desired, nucleation initiators such as seed crystals can be added to induce nucleation or an antisolvent can be added to induce precipitation. The presence of solid forms is then determined, for example, by optical detection, and the solvent removed by filtration or evaporation. The crystal properties, such as polymorph or habit can then determined using techniques such as Raman, melting point, x-ray diffraction, and the like, with the results of the analysis being analyzed using an appropriate data processing system.

Additionally, the computer-controlled system can include a variety of features for implementing the computer-controlled methods, which can be implemented by a computer-program product operating in the computing system. As such the computing system and/or computer-program product can include a database comprising at least one table that has at least one of the following: (a) a plurality of molecular descriptors; (b) a plurality of compound identifiers; (c) a plurality of compound/descriptor relations associating compound identifiers with molecular descriptors; (d) a plurality of empirically determined physical, chemical and biological parameters; (e) a plurality of compound/parameter relations associating compound identifiers with the empirically determined physical, chemical and biological parameters; and (f) data representing results from a plurality of experiments performed with a high-throughput automated system. Additionally, the computing system and/or computer-program product can include a query system for selecting subsets of related information from the at least one table. Further, the computing system and/or computer-program product can include a multidimensional representation generation module capable of generating visual representations of data sets having at least four dimensions. Furthermore, the computing system and/or computer-program product can include a plurality of modeling modules, each module being capable of receiving information selected by the query system and estimating at least one property of a multi-component chemical composition.

An embodiment of a computer-controlled system is described in more detail below with references to FIGS. 2A-2C. FIG. 2A is a schematic overview of a high-throughput system for generation and analysis of approximately 25,000 solid forms of an active component and shows the overall system, which consists of a series of integrated modules, or workstations. Functionally, the system consists of three main modules: sample generation 10, sample incubation 30, and sample detection 50.

As shown in more detail in FIG. 2B, the sample generation module 10 begins with labeling and identification of each plate 14 (for example, using high speed inkjet labeling 16 and bar-code reading 18). Once labeled, the plates 14 proceed to the dispensing sub-modules. The first dispensing sub-module 20 is where the compound(s)-of-interest are dispensed into the sample wells or sample tube of the plates. Additional dispensing sub-modules 22a, 22b, 24a, and 24b are employed to add compositional diversity. Note there is a minimum of one dispenser in each of these sub-modules, but there can be as many as is practical. One sub-module 22a can dispense antisolvent to the sample solution. Another sub-module 22b can dispense additional reagents, such as surfactants, crystallizing aids, and the like, in order to enhance crystallization. A critical component of one of the sub-modules 24a or 24b is the ability to dispense sub-microliter amounts of liquid. This nanoliter dispensing can involve the use of inkjet technology (in any of its forms) and is preferably compatible with organic solvents. If desired, after dispensing is complete, the plates can be sealed to prevent solvent evaporation. The sealing mechanism 26 can be a glass plate with an integrated chemically compatible gasket (not shown). This mode of sealing allows optical analysis of each sample site without having to remove the seal.

The sealed plates 28 from the sample generation module next enter into the sample incubation module 30, shown in FIG. 2C. The incubation module 30 consists of four sub-modules. The first sub-module is a heating chamber 32. In one example of use of the incubation chamber, the sample plates can be heated to a temperature (T1). This heating dissolves any compounds that may have undergone precipitation in the previous process. After incubating at this elevated temperature for a period of time, each well (not shown) can be analyzed for the presence of undissolved solids. Wells that contain solids are identified and can be filtered or tracked throughout the process in order to avoid being deemed a “hit” in the final analysis. After the heating treatment, the plates can be subjected to a cooling treatment to a final temperature T2, using cooling sub-module 34. Preferably, this cooling sub-module 34 maintains uniform temperature across each plate in the chamber (+−1° C.). At this point, if desired, the samples can be subjected to a nucleating event from nucleation station 33. Nucleation events include mechanical stimulation and exposure to sources of energy, such as acoustic (e.g. ultrasound), electrical, or laser energy. A nucleation event also includes addition of nucleation promoters or other components, such as additives, that decrease the surface energy or seed crystals of the compound of interest. During cooling, each sample is analyzed for the presence of solid formation. This analysis allows the determination of the temperature at which crystallization or precipitation occurred.

III. Preparing and Processing Arrays of Samples

The computer-controlled automated high-throughput system and/or computer-program products operating in the computing system can be used for designing, preparing, processing, screening, and analyzing samples having experimental formulations comprising a compound of interest. After the experimental formulation for each sample has been designed by the computer-controlled system and/or computer-program products, the automated high-throughput system can prepare the array of samples. As such, compound of interest and any additional components can be delivered to a plurality of sample sites in an array, such as sample wells or sample tubes on a sample plate to give an array of unprocessed samples. The array can then be processed according to the purpose and objective of the experiment, and one of skill in the art will readily ascertain the appropriate processing conditions. Preferably, the automated distribution mechanism as described above is used to distribute or add components.

The array can be processed by the computer-controlled system according to the design and objective of the experiment. One of skill in the art will readily ascertain the appropriate processing conditions. Processing includes mixing; agitating; heating; cooling; adjusting the pressure; adding additional components, such as crystallization aids, nucleation promoters, nucleation inhibitors, acids, or bases, and the like, stirring, milling, filtering, centrifuging, emulsifying, subjecting one or more of the samples to mechanical stimulation, ultrasound or laser energy, or subjecting the samples to temperature gradiation or simply allowing the samples to stand for a period of time at a specified temperature. A few of the more important processing parameters are elaborated below.

A. Temperature

In some array experiments, processing will comprise dissolving either the compound of interest or one or more components. Solubility is commonly controlled by the composition (identity of components and/or the compound of interest) or by the temperature. The latter is most common in industrial crystallizers where a solution of a substance is cooled from a state in which it is freely soluble to one where the solubility is exceeded. For example, the array can be processed by heating to a temperature (T1), preferably to a temperature at which the all the solids are completely in solution. The samples are then cooled, to a lower temperature (T2). The presence of solids can then be determined. Implementation of this approach in arrays can be done on an individual sample site basis or for the entire array (i.e., all the samples in parallel). For example, each sample site could be warmed by local heating to a point at which the components and the compound of interest are dissolved. This step is followed by cooling through local thermal conduction or convection. A temperature sensor in each sample site can be used to record the temperature when the first crystal or precipitate is detected.

In one embodiment, all the sample sites are processed individually with respect to temperature and small heaters, cooling coils, and temperature sensors for each sample site are provided and controlled. This approach is useful if each sample site has the same composition and the experiment is designed to sample a large number of temperature profiles to find those profiles that produce desired solid forms. In another embodiment, the composition of each sample site is controlled and the entire array is heated and cooled as a unit. The advantage of the latter approach is that much simpler heating, cooling, and controlling systems can be utilized. Alternatively, thermal profiles are investigated by simultaneous experiments on identical array stages. Thus, a high-throughput matrix of experiments in both composition and thermal profiles can be obtained by parallel operation.

Typically, several distinct temperatures are tested during crystal nucleation and growth phases. Temperature can be controlled in either a static or dynamic manner. Static temperature means that a set incubation temperature is used throughout the experiment. Alternatively, a temperature gradient can be used. For example, the temperature can be lowered at a certain rate throughout the experiment. Furthermore, temperature can be controlled in a way as to have both static and dynamic components. For example, a constant temperature (e.g. 60° C.) is maintained during the mixing of crystallization reagents. After mixing of reagents is complete, controlled temperature decline is initiated (e.g. from 60° C. to about 25° C. over 35 minutes).

B. Time

Array samples can be incubated or processed for various lengths of time (e.g. 5 minutes, 60 minutes, 48 hours, and the like). Since phase changes can be time dependent, it can be advantageous to monitor array experiments as a function of time. In many cases, time control is very important; for example, the first solid form to crystallize may not be the most stable, but rather a metastable form which can then convert to a form stable over a period of time. This process is called “ageing”. Ageing also can be associated with changes in crystal size and/or habit. This type of ageing phenomena is called Ostwald ripening.

C. pH

The pH of the sample medium can determine the physical state and properties of the experimental formulation as generated. The pH can be controlled or changed by the addition of inorganic and organic acids and bases. The pH of samples can be monitored with standard pH meters modified according to the volume of the sample.

D. Concentration

The concentration of the compound of interest and/or any additional component can determine the chemical and/or physical state and properties of the experimental formulation that is generated. The concentration of the compound of interest and/or any additional component can be controlled or changed by the amount added to each experimental formulation.

In some instances, it can be preferred that the compound of interest be formulated at a concentration above saturation or at supersaturation. Supersaturation is the thermodynamic driving force for both crystal nucleation and growth and thus is a key variable in processing arrays. Supersaturation is defined as the deviation from thermodynamic solubility equilibrium. Thus, the degree of saturation can be controlled by temperature and the amounts or concentrations of the compound of interest and other components. In general, the degree of saturation can be controlled in the metastable region, and when the metastable limit has been exceeded, nucleation will be induced.

The amount or concentration of the compound of interest and components can greatly affect physical state and properties of the resulting solid form. Thus, for a given temperature, nucleation and growth will occur at varying amounts of supersaturation depending on the composition of the starting solution. Nucleation and growth rate increase with increasing saturation, which can affect crystal habit. For example, rapid growth must accommodate the release of the heat of crystallization. This heat effect is responsible for the formation of dendrites during crystallization. The macroscopic shape of the crystal is profoundly affected by the presence of dendrites and even secondary dendrites. For example, the first crystal to be formed from a concentrated solution is formed at a higher temperature than that formed from a dilute solution. The second effect that the relative amounts compound of interest and solvent has is the chemical composition of the resulting solid form. Thus, the equilibrium solid phase is that from a higher temperature in the phase diagram. Thus, a concentrated solution may first form crystals of the hemihydrate when precipitated from aqueous solution at high temperature. The dihydrate may, however, be the first to form when starting with a dilute solution. In this case, the compound of interest/solvent phase diagram is one in which the dihydrate decomposes to the hemihydrate at a high temperature. This is normally the case and holds for commonly observed solvates.

E. Identity of the Components

The identity of the components in the sample medium has a profound effect on almost all aspects of solid formation. Component identity will affect (promote or inhibit) crystal nucleation and growth as well as the physical state and properties of the resulting solid forms. Thus, a component can be a substance which has the intended effect in an array sample to induce, inhibit, prevent, or reverse formation of solid forms of the compound of interest. A component can direct formation of crystals, amorphous-solids, hydrates, solvates, or salt forms of the compound of interest. Components also can affect the internal and external structure of the crystals formed, such as the polymorphic form and the crystal habit. Examples of components include, but are not limited to, excipients; solvents; salts; acids; bases; gases; small and large molecules; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid forms; additives that dissolve solid forms; additives that inhibit crystallization or solid formation; optically-active solvents; optically-active reagents; and optically-active catalysts.

F. Solvent

In general, arrays of the invention will contain a solvent as one of the components. Solvents may influence and direct the formation of solid forms through polarity, viscosity, boiling point, volatility, charge distribution, and molecular shape. The solvent identity and concentration is one way to control saturation. Indeed, one can crystallize under isothermal conditions by simply adding a nonsolvent (i.e., antisolvent) to an initially subsaturated solution. One can start with an array of a solution of the compound of interest in which varying amounts of nonsolvent are added to each of the individual elements of the array. The solubility of the compound is exceeded when some critical amount of nonsolvent is added. Further addition of the nonsolvent increases the supersaturation of the solution and, therefore, the growth rate of the crystals that are grown. Mixed solvents also add the flexibility of changing the thermodynamic activity of one of the solvents independent of temperature. Thus, one can select which hydrate or solvate is produced at a given temperature simply by carrying out crystallization over a range of solvent compositions. For example, crystallization from a methanol-water solution that is very rich in methanol will favor solid form hydrates with fewer waters incorporated in the solid (e.g. dihydrate vs. hemihydrate) while a water-rich solution will favor hydrates with more waters incorporated into the solid. The precise boundaries for producing the respective hydrates are found by examining the elements of the array when concentration of the solvent component is the variable.

The use of different solvents or mixtures of solvents will influence the solid forms that are generated. Solvents may influence and direct the formation of the solid phase through polarity, viscosity, boiling point, volatility, charge distribution, and molecular shape. In a preferred embodiment, solvents that are generally accepted within the pharmaceutical industry for use in manufacture of pharmaceuticals are used in the arrays. Various mixtures of those solvents can also be used. The solubilities of the compound of interest can be high in some solvents and low in others. Solutions can be mixed in which the high-solubility solvent is mixed with the low-solubility solvent until solid formation is induced. Hundreds of solvents or solvent mixtures can be screened to find solvents or solvent mixtures that induce or inhibit solid form formation. Solvents include, but are not limited to, aqueous-based solvents such as water or aqueous acids, bases, salts, buffers or mixtures thereof and organic solvents, such as protic, aprotic, polar or non-polar organic solvents.

G. Control of Solvent-Removal Rate

Control of solvent removal is intertwined with control of saturation. As the solvent is removed, the concentration of the compound of interest and less-volatile components becomes higher. And depending on the remaining composition, the degree of saturation will change depending on factors such as the polarity and viscosity of the remaining composition. For example, as a solvent is removed, the concentration of the component-of-interest can rise until the metastable limit is reached and nucleation and crystal growth occur. The rate of solvent removal can be controlled by temperature and pressure and the surface area under which evaporation can occur. For example, solvent can be removed by distillation at a predefined temperature and pressure, or the solvent can be removed simply by allowing the solvent to evaporate at room temperature.

H. Inducing Nucleation or Precipitation

Once an array is prepared, solid formation can be induced by introducing a nucleation or precipitation event. In general, this involves subjecting a supersaturated solution to some form of energy, such as ultrasound or mechanical stimulation, or by inducing supersaturation by adding additional components.

1. Inducing a Nucleation Event

Crystal nucleation is the formation of a crystal solid phase from a liquid, an amorphous phase, a gas, or from a different crystal solid phase. Nucleation sets the character of the crystallization process and is therefore one of the most critical components in designing commercial crystallization processes. So called primary nucleation can occur by heterogeneous or homogeneous mechanisms, both of which involve crystal formation by sequential combining of crystal constituents. Primary nucleation does not involve existing crystals of the compound of interest, but results from spontaneous formation of crystals. Primary nucleation can be induced by increasing the saturation over the metastable limit or, when the degree of saturation is below the metastable limit, by nucleation. Nucleation events include mechanical stimulation, such as contact of the crystallization medium with the stirring rotor of a crystallizer and exposure to sources of energy, such as acoustic (ultrasound), electrical, or laser energy. Primary nucleation can also be induced by adding primary nucleation promoters. That is, substances other than a solid form of the compound of interest.

Secondary nucleation involves treating the crystallizing medium with a secondary nucleation promoter that is a solid form; preferably, a crystalline form of the compound of interest. Direct seeding of samples with a plurality of nucleation seeds of a compound of interest in various physical states provides a means to induce formation of different solid forms. In one embodiment, particles are added to the samples. In another, nanometer-sized crystals (nanoparticles) of the compound of interest are added to the samples.

2. Inducing a Precipitation Event

The term precipitation is usually reserved to describe the formation of an amorphous solid or semi-solid from a solution phase. Precipitation can be induced in much the same way as discussed above for nucleation the difference being that an amorphous rather than a crystalline solid is formed. Addition of a nonsolvent to a solution of a compound of interest can be used to precipitate a compound. The nonsolvent rapidly decreases the solubility of the compound in solution and provides the driving force to induce solid precipitate. This method generally produces smaller particles (higher surface area) than by changing the solubility in other ways, such as by lowering the temperature of a solution. The invention provides means to identify the optimal solvents and solvent concentrations for providing an optimal solid form or for preventing formation or inducing solvation of a solid form. The invention can be used to greatly speed the process of identifying useful precipitation solvents.

IV. Screening Experimental Formulations

The experimental formulations can be screened by various techniques in order to identify the changes in the chemical and/or physical properties of the compound of interest or experimental formulation. Some screening techniques can be performed on experimental formulations that contain a solid or are completely solid. Some preferred examples of screening techniques are described below.

In certain embodiments, after processing, samples can be analyzed to detect the presence or absence of solid forms, and any solid forms detected can be further analyzed to characterize the properties and physical state.

Advantageously, samples in commercially available microtiter plates can be screened for the presence or absence of solids (e.g., precipitates or crystals) using automated plate readers. Automated plate readers can measure the extent of transmitted light across the sample. Diffusion (reflection) of transmitted light indicates the presence of a solid form. Visual or spectral examination of these plates can also be used to detect the presence of solids. In yet another method to detect solids, the plates can be scanned by measuring turbidity.

If desired, samples containing solids can be filtered to separate the solids from the medium, resulting in an array of filtrates and an array of solids. For example, the filter plate comprising the suspension is placed on top of a receiver plate containing the same number of sample wells, each of which corresponds to a sample site on the filter plate. By applying either centrifugal or vacuum force to the filter plate over receiver plate combination, the liquid phase of the filter plate is forced through the filter on the bottom of each sample well into the corresponding sample well of the receiver plate. A suitable centrifuge is available commercially, for example, from DuPont, Wilmington, Del. The receiver plate is designed for analysis of the individual filtrate samples.

After a solid is detected it can be further analyzed to define its physical state and properties. In one embodiment, on-line machine vision technology is used to determine both the absence/presence of crystals as well as detailed spatial and morphological information. Crystallinity can be assessed and distinguished from amorphous solids automatically by using commercially available plate readers with a polarized filter apparatus to measure the total light to determine crystal birefringence; crystals turn polarized light, while amorphous materials absorb the light. It is also possible to monitor turbidity or birefringence dynamically throughout the crystal forming process.

Examples of analytical techniques that can analyze solid formulations can include Raman spectroscopy, infrared spectroscopy, second harmonic generation, x-ray crystallography, X-ray powder diffraction, image-analysis, microscopy, photomicrography, optical-image analysis, electron microscopy, scanning electron microscopy (SEM), transmission electron microscope (TEM), near-field scanning optical microscopy (NSOM or SNOM), far-field scanning optical microscopy (FSOM), atomic force microscopy (AFM), micro-thermal analysis (Micro-TA), differential thermal analysis (DTA), differential scanning calorimetry (DSC), and the like.

L. Analytical Methods Requiring Dissolution of the Sample

While in some cases it is necessary to analyze the products of a solid-state reaction in the solid without dissolution, many of the most popular analytical methods of analysis require dissolution of the sample. These analytical techniques are useful for solid-state reactions if the reactants and products are stable in solution. For example, for solid-state reactions induced by heat or light, it is convenient to remove the heat or light, dissolve the sample, and analyze the products. Such analytical techniques can include ultraviolet spectroscopy, nuclear magnetic resonance (NMR), gas chromatography, high-pressure liquid chromatography (HPLC), thin-layer chromatography (TLC), and the like.

V. Directed Search Strategy

The present invention can include a computer-controlled automated high-throughput system to implement a directed search strategy for determining a multi-component chemical composition. The directed search strategy can be employed in an experimentation method that includes designing, preparing, and testing a first array, and subsequently using data from the first array to design, prepare, and test a second array. In some instances, the directed search strategy can be used as iterations in identifying at least one optimal formulation. In some instances, the directed search strategy can be used to study a first set of variables, and the data from the first set of variables can then be used to study a second set of variables. In part, this can include first studying the effects of the identity of the additional components or distinct combinations of additional components, and then using data obtained from the additional components to study concentration gradients of selected components. Also, the computer-controlled system can implement a method for determining at least one solid form of a compound for a desired use, wherein the solid form can be an optimal solid form.

Accordingly, the system and method can include selecting and inputting a combination of experimental parameters that may be varied by the computer-controlled system. The computing system can determine a first plurality of distinct combinations of values for each of the experimental parameters, wherein each combination corresponds to a distinct experiment. Each distinct experiment can include a distinct experimental formulation designed by the computing system and having the component of interest and additional components formulated in accordance with the distinct combinations of values. The computer-controlled system can conduct a first set of experiments after each experimental formulation is prepared in an array of samples, wherein each experiment of the first set can correspond to a distinct combination of values of the first plurality of distinct combinations. The computing system can process and analyze the experimental formulations in order to determine a first collection of experimental results for the first set of experiments. The first collection of experimental results can include a plurality of individual result sets that, in turn, each correspond to a distinct experiment as described above.

Based on the first collection of experimental results, the computing system can thereafter determine a second plurality of distinct combinations of values of experimental parameters to be varied by the computer-controlled system. Each of the second plurality of distinct combinations of values of experimental parameters can correspond to a distinct experiment. Each distinct experiment can include a distinct experimental formulation designed by the computing system and having the component of interest and additional components formulated in accordance with the distinct combinations of values. The computer-controlled automated system can conduct a second set of experiments after each experimental formulation is prepared in an array of samples, wherein each experiment of the second set can correspond to a distinct combination of values of the second plurality of distinct combinations. The computing system can process and analyze the experimental formulations in order to determine a second collection of experimental results of the second set of experiments. The second collection of experimental results can include a plurality of individual result sets that in turn each correspond to a distinct experiment. The computing system can then select at least one multi-component experimental formulation based on the first collection of experimental results and the second collection of experimental results. Also, the computing system can include a computer-program product for selecting at least one experimental formulation based on the first collection of experimental results and the second collection of experimental results.

In one embodiment, the present invention can include implementation by the computer-controlled automated high-throughput system of a method of using algorithms to analyze data in determining at least one multi-component chemical composition for a desired use. In some instances, the chemical composition can include a solid form of a compound of interest. The computing system can be used for designing and conducting a plurality of experiments on an array of samples. The computing system can analyze the plurality of experiments in order to obtain data for each experiment. The data can then be stored in the computing system or a databank associated with the computing system. As such, the data can represent a set of experimental parameters, a set of experimental results, and/or a set of molecular descriptors characterizing an aspect of the experiment. The computing system can then associate the experimental data from the plurality of experiments with previously stored data by querying a database comprising information not derived from the plurality of experiments. The information from the database and the experimental data can then be processed by the computing system by processing the experimental data with a processor that is programmed to apply a discriminator algorithm to associate at least one experiment with at least one classification. As such, the computing system can include a computer-program product for using algorithms to analyze the data.

In one embodiment, the computing system and/or computer-program product can be used in a method for selecting a compound of interest for further testing. Such a method can include receiving information or experimental data regarding a plurality of compounds of interest and performing high-throughput design, preparation, and screening of at least one of the plurality of compounds of interest to identify at least one optimal formulation, which can include a solid form of the compound of interest. At least one of the plurality of compounds of interest can be selected for further testing based on at least one property of each identified optimal formulation.

In one embodiment, the computing system and/or computer-program product can be used in a method for selecting a form of a compound, such as a solid form, for further testing. The method can include receiving information or experimental data for a compound, and performing high-throughput solid form design, preparation, and screening to identify at least two forms of the compound. At least one form of the compound of interest can be selected for further testing based on at least one property of each identified optimal formulation.

In one embodiment, the computing system and/or computer-program product can be used in a method for selecting a formulation of a compound, such as a solid form, for further testing. The method can include receiving information or experimental data for a compound, and performing high-throughput solid form designing, preparing, and screening to identify at least one formulation of the compound. At least one formulation of the compound of interest can be selected for further testing based on at least one property of each identified optimal formulation.

In one embodiment, the computing system and/or computer-program product can be used in a method for determining whether to further test at least one compound. The method can include receiving information or experimental data for a compound, and performing high-throughput solid form design, preparation, and screening to identify at least one formulation of the compound having a selected property. At least one formulation of the compound of interest can be selected for further testing based on the selected property of each identified optimal formulation.

Accordingly, the computer-controlled automated high-throughput system and/or computer-program product can be used in methods which may be used to prioritize testing procedures or direct testing to be completed in a series of steps. As such, the series of experiments can be used to study the concentration of the compound of interest, concentration of components in the experimental formulations, identity of components, combination of components, additive, solvent, antisolvent composition, temperature, temperature change, heating, cooling, nucleation seeds, supersaturation, pH, pH change, and time of crystallization reaction by studying one type of variable in each assay. As such, a series of assays using data from previous assays can incrementally identify formulations having desired chemical and/or physical properties.

In one embodiment, a computer-controlled system designed for controlling automated high-throughput processing of an array having a large number of samples can be used in a directed search strategy to identify chemical and/or physical properties leading to optimal formulation for a given use of a compound of interest. The computing system can provide computer-aided design and processing of an experimental formulation for each sample. Each experimental formulation can have the compound of interest and can be based on at least one experimental variable which is varied as to at least some samples so that the effect in terms of changes in the chemical and/or physical properties of the compound of interest due to at least one experimental variable can be identified across a large number of comparative samples.

A method of using the computer-controlled system for implementing a directed search strategy can include the following: inputting into the computing system at least one compound of interest and any additional components to be included in a plurality of experimental formulations that are to be designed for a first array of samples; inputting into the computing system at least one selected experimental variable of interest that is to be varied as between at least some samples of the first array; the computing system thereafter designing a plurality of unique experimental formulations that differ as between at least some samples based on the at least one selected experimental variable of interest that is varied as between the at least some samples of the first array; the computing system thereafter controlling a process by which an experimental formulation for each sample is prepared and tested in order to create changes in chemical and/or physical properties across a large number of comparative samples for the at least one compound of interest; inputting into the computing system detected changes across the large number of comparative samples for the at least one compound of interest; the computing system thereafter screening the large number of samples by identifying those samples which contain chemical and/or physical properties likely to lead to an optimal formulation for a given use of a compound of interest, and storing as a first data set information as to the experimental formulation and the resulting chemical and/or physical properties for each of the identified samples; inputting to the computing system at least one other selected experimental variable of interest that is to be varied as between at least some identified samples of the first data set; the computing system thereafter designing a plurality of further experimental formulations for a second array having a large number of samples that are different as between at least some of the identified samples of the first data set based on the at least one further selected experimental variable of interest that is to be varied as between the at least some identified samples of the first data set; the computing system thereafter controlling a process by which the plurality of further experimental formulations in the second array of samples are prepared and tested in order to create further changes in chemical and/or physical properties across further comparative samples for the at least one compound of interest; inputting into the computing system detected further changes across the further comparative samples of the first data set for the at least one compound of interest; the computing system thereafter screening the further comparative samples by identifying changes in chemical and/or physical properties and storing as a second data set information as to the plurality of further experimental formulations and the resulting chemical and/or physical properties for each further comparative sample; and the computing system thereafter selecting from the first and second data sets those samples which contain chemical and/or physical properties likely to lead to an optimal formulation for a given use of a compound of interest.

In one embodiment, a computer-program product can comprise a computer-readable medium containing computer-executable instructions for causing the computing system to execute a directed search strategy method. Any known computer-readable medium can be used, examples of which include optical disks, magnetic disks, magnetic tape, flash memory, and the like.

In the directed search strategy, at least one selected experimental variable of interest and at least one further experimental variable of interest that are to be varied as between at least some samples of the array, are each varied as to at least one of concentration of the compound of interest, concentration of components in the experimental formulations, identity of components, combination of components, additive, solvent, antisolvent composition, temperature, temperature change, heating, cooling, nucleation seeds, supersaturation, pH, pH change, or time of crystallization reaction.

Additionally, the directed search strategy can analyze the experimental formulations for chemical and/or physical properties likely to lead to optimal formulation for a given use of a compound of interest. The chemical and/or physical properties can include microstructure, crystallinity, amorphism, polymorphism, hydrate, solvate, isomorphic desolvate, packing order, ionic crystal, interstitial space, lattice, or habit.

The directed search strategy can include inputting into the computing system a data set, based on analyzing the preparation and processing of each of the experimental formulations in the array of sample, having experimental data for the changes across the large number of comparative samples or further comparative samples for the at least one compound of interest. The data set can then be analyzed to determine at least one optimal formulation for a given use of a compound of interest.

In the directed search strategy, the computing system can at least partially control or assist in screening the chemical and/or physical properties of each of the experimental formulations in the array of samples for at least one desired property. Also, the computing system can at least partially control or assist in identifying at least one experimental formulation having the at least one desired property.

Also, a first set of the plurality of further experimental formulations in the second array of samples can have different concentrations of at least one additional component in at least one experimental formulation of the first array of samples. The selected experimental variable of interest can include the identity of any additional components. The further selected experimental variable of interest can include a concentration gradient for at least one selected additional component. Also, the further selected experimental variable of interest can include a concentration gradient for the at least one compound of interest.

In one embodiment of the present invention, the computer-controlled automated high-throughput system can be used in conjunction with one or more high-throughput automated experimentation apparatus, such as Transform Pharmaceutical's FAST™ formulation system or CRYSTALMAX™ crystal discovery system and can function as a directed search system. The FAST and CRYSTALMAX systems are described in U.S. patent application Ser. Nos. 09/628,667 and 09/756,092, respectively, (the “FAST” and “CRYSTALMAX” applications) which are incorporated herein by reference. The computer-controlled system is used to plan, prepare, perform, screen, and analyze experiments performed with the CRYSTALMAX and FAST systems. Additionally, the descriptions of the following computer-controlled systems can be used with other embodiments of the invention in addition to directed search strategies.

Accordingly, the computer-controlled system can include a process informatics subsystem for controlling and acquiring data from the CRYSTALMAX and FAST systems, and a computational informatics subsystem for performing data mining, simulation, molecular modeling, high-dimensional multivariate visualizations of data, data clustering, categorizations, and other data processing. These subsystems can operate on a shared database system used to store experimental results and analyses, as well as data derived from sources other than the process informatics subsystem, such as external databases and literature.

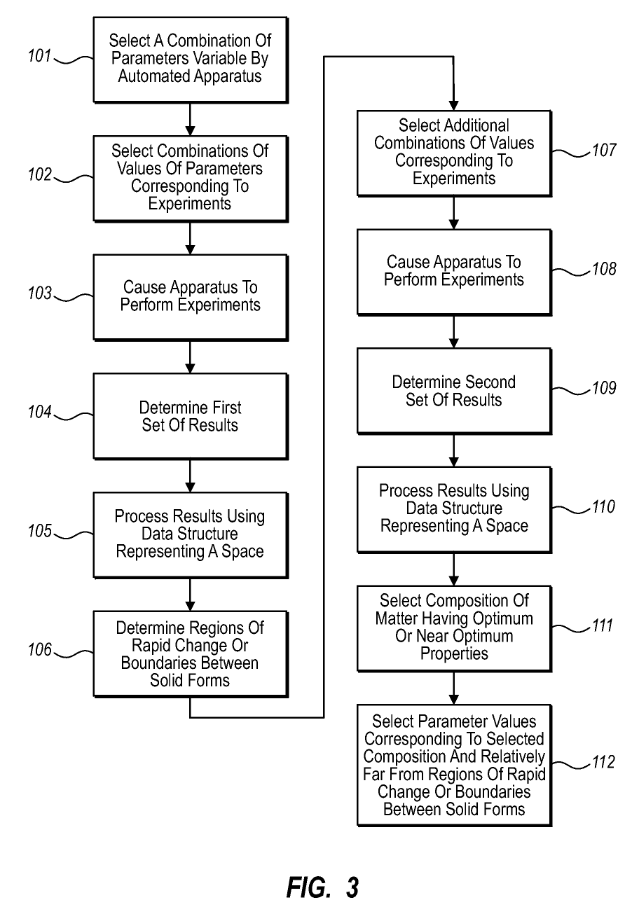

As schematically illustrated in FIG. 3, using the computational informatics subsystem, a combination of experimental parameters which may be varied by an automated experimentation apparatus, such as FAST or CRYSTALMAX, is selected 101. A first plurality of distinct combinations of values of the experimental parameters is then determined, each combination corresponding to a distinct experiment 102. Using the process informatics subsystem, the automated experimentation apparatus is caused to conduct a first set of experiments, each experiment of the first set corresponding to a distinct combination of the first plurality of distinct combinations 103. The process informatics subsystem is also used to determine a first collection of experimental results of the first set of experiments, the first collection comprising a plurality of individual result sets, where each individual result set corresponds to a distinct experiment 104.

The first collection of experimental results can be processed through the computational informatics subsystem to determine a second plurality of distinct combinations of values of the experimental parameters, each combination of the second plurality corresponding to a distinct experiment.

Preferably, data representing the first collection of experimental results is processed as a collection of points in a space, such as a topological space, a metric space, or a vector space, comprising dimensions corresponding to the dimensions of the experimental parameters 105. Through such analysis, regions of the space are determined in which significant changes in result sets occur in connection with relatively small changes in the experimental parameters. For example, boundaries between solid forms, or regions in which desired properties of formulations change rapidly with experimental parameters, are preferably identified 106. Based on this identification, the second plurality of distinct combinations of values of the experimental parameters is preferably selected 107 to more fully define such boundaries or regions, and to include combinations of parameters as far as possible from such boundaries or regions.

Using the process informatics subsystem, the computer-controlled system apparatus is activated to conduct a second set of experiments, each experiment of the second set corresponding to a distinct combination of values of the second plurality 108. The process informatics subsystem is also used to determine a second collection of experimental results of the second set of experiments, the second collection comprising a plurality of individual results, each individual result corresponding to a distinct experiment 109.

The computational informatics subsystem is then used to select a multi-component chemical composition of matter based on the first collection of experimental results and the second collection of experimental results. Alternatively, additional iterations of experimentation may be performed prior to selecting the multi-component chemical composition.

As with the prior collection of experimental results, data representing the second or subsequent collection of experimental results is preferably processed as a collection of points in a space such as topological space, metric space, or vector space comprising dimensions corresponding to the dimensions of the experimental parameters 110. Based on this processing, a set of experimental parameter values and a resulting multi-component chemical composition of matter is preferably selected having optimum or near-optimum properties that do not change significantly within a region of the space corresponding to an expected range of conditions of manufacture, storage, and administration or use 111.

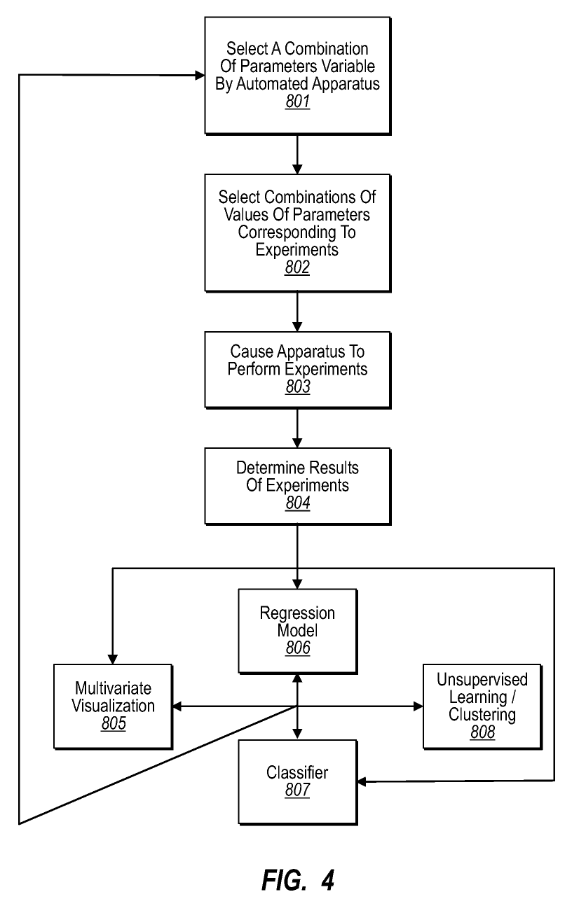

FIG. 4 illustrates another embodiment of a directed search strategy that can be implemented on a computer-controlled automated high-throughput system in accordance with the present invention. As such, the first collection of experimental results is processed through the computational informatics subsystem to determine a second combination of parameters variable by the computer-controlled system 801, and a second plurality of distinct combinations of values of the experimental parameters 802, each combination of the second plurality corresponding to a distinct experiment. This process preferably may be iterated indefinitely to yield a third, fourth, fifth, or arbitrary number of subsequent pluralities of distinct combinations of experimental parameters, each combination corresponding to a distinct experiment. Although each combination preferably corresponds to a distinct experiment, in some circumstances multiples of each experiment are preferably performed to provide reliable data, particularly in stochastic processes such as crystallization.

To determine combinations of parameters and values of the parameters, one or more multivariate visualizations 805, generated models 806 and 807, and/or unsupervised learning or clustering methods 808 are preferably employed. Generated models preferably comprise one or more regression model 806 and/or one or more classification model 807. A classification model takes one or more inputs and provides at least one class assignment as an output. A regression model takes one or more inputs and provides at least one output representing a variable that has a continuous range (e.g. at least one real or complex interval). The foregoing are preferably employed in combination, for example, a multivariate visualization of the results of a clustering calculation may be used to determine a classifier, as described more fully below.

The following exemplary classification and regression models in planning and assessing experiments to determine formulations and solid forms illustrate some of the ways in which each type of model may be used. A classification model comprising a qualitative solubility assay may, for example, be used in conjunction with the FAST system to assign a soluble/not soluble label to each individual experimental result set. A regression model comprising a quantitative solubility assay may, for example, be used with FAST to assign an estimated solubility, expressed for example in mg/ml. In conjunction with the CRYSTALMAX system, a classification model may, for example, be used to assign a polymorph label to each individual experimental result set producing a solid form. A regression model may be used with CRYSTALMAX to, for example, provide an estimated nucleation time. For each model, the input may comprise experimental parameters and/or results.

Regression models may include, but are not limited to linear regression, stepwise linear regression, additive models (AM), projection pursuit regression (PPR), recursive partitioning regression (RPR), alternating conditional expectations (ACE), additivity and variance stabilization (AVAS), locally weighted regression (LOESS), neural networks, Multivariate Adaptive Regression Splines (MARS), principal components regression, partial least squares regression, and support vector regression. Many other regression methods may be found in the literature.

Classification models may include, but are not limited to, decision trees (e.g., generated by algorithm like C4.5, C5.0, or CART), support vector machines, neural networks, k-nearest neighbor classifiers, Bayesian classifiers (with probability density functions preferably determined using Gaussian Mixture Models or Parzen windowing), self-organizing maps.

One or more models may preferably be generated based on the results of unsupervised learning and/or clustering applied to one or more collections of experimental result sets. In one preferred embodiment, described more fully below, a collection of individual experimental result sets is received, a similarity measure is calculated between a plurality of pairs of individual experimental result sets, and based on the similarity measure, a plurality of clusters of experimental result sets is determined, and one or more properties is determined for at least one solid form from each of at least two of the clusters. A three-dimensional visualization is preferably used to display the clusters. Preferably, each experimental result set in each cluster corresponds to a single solid form, preferably a single crystal polymorph. By characterizing the solid form corresponding to each cluster, solid form labels may be determined for each experimental result set for each cluster. Based on these labels and the experimental result sets and experimental parameters, a classifier model and/or a regression model may generated.

Unsupervised learning and clustering methods may include hierarchical clustering, including agglomerative and stepwise-optimal hierarchical clustering, k-means clustering, Gaussian mixture model clustering, or self-organizing-map (SOM)-based clustering, clustering using the Chameleon, DBScan, CURE, or Rock clustering algorithms, unsupervised Bayesian learning, Principal Component Analysis, Nonlinear Component Analysis, Independent Component Analysis, and multidimensional scaling.

In one embodiment, the experimental result sets comprise Raman spectra, the similarity measure comprises the Tanimoto distance between bit-vectors representing peaks in Raman spectra, and the clustering method comprises hierarchical k-means clustering. The results of the preferred hierarchical clustering of Raman spectra described above are preferably displayed using a three-dimensional representation (two spatial coordinates plus color or shading).

Based on the one or more generated models and/or multivariate visualizations, additional combinations of experimental parameters can be determined to meet one or more experimental objectives. The experimental objectives preferably include determining boundaries between solid forms, determining regions in which desired properties of formulations change rapidly with respect to changes in experimental parameters (not necessarily with respect to time), extrema (e.g. maxima or minima) of experimental results or parameters, regions within a class boundary, or regions of ambiguity or low confidence in classification or regression results.

VI. Planning and Assessing a Massively Parallel Search for New Solid Forms

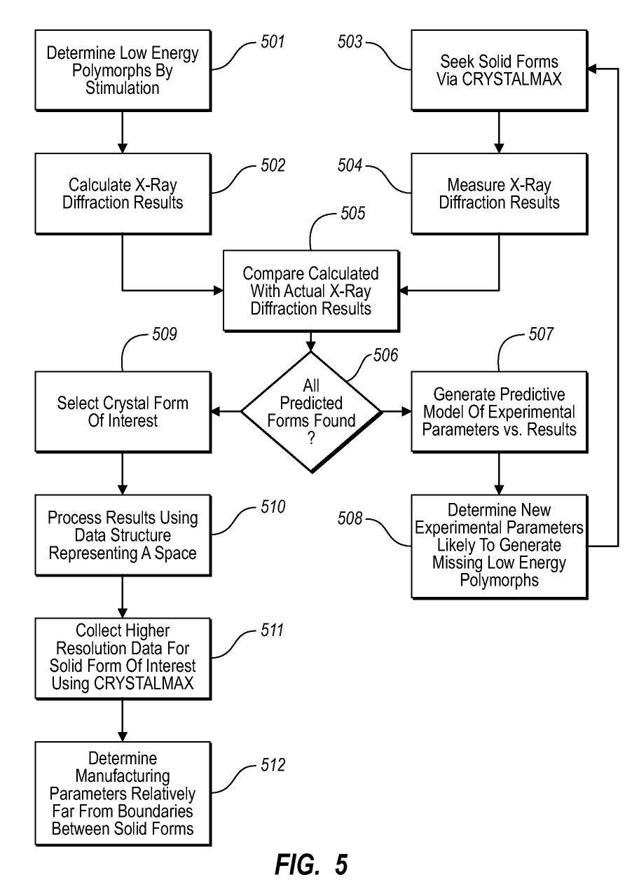

In one embodiment, the present invention includes a method to assess the first collection of experimental results in a search for novel or known solid forms is schematically illustrated in FIG. 5. The method comprises the steps of: determining low-energy crystal polymorphs via simulation 501; characterizing the low-energy crystal polymorphs according to expected experimental results by standard techniques such as by calculated X-ray powder or single-crystal diffraction results 502; conducting a first collection of crystallization experiments 503; measuring a collection of actual experimental results such as actual X-ray powder diffraction for the crystals produced by the first collection of crystallization experiments 504; comparing the expected experimental results with the actual experimental results 505; determining if any lowest-energy structures were not included in the solid forms produced by a first collection of experiments 506.

Preferably, low-energy polymorphs are determined by using multivariate optimization such as hydrogen-bond-biased simulated annealing to locate a plurality of lowest-energy structures with the model. One preferred energy function is crystal lattice energy, also referred to as the crystal binding or cohesive energy. Lattice energy is determined by summing all the pairwise atom-atom interactions between a central molecule and all the surrounding molecules. The lattice energy is a useful parameter because its calculated value can be compared with the experimental enthalpy of sublimation. This allows one to verify the description of the intermolecular interactions by the force field in question.

An advantage of the calculated value of the crystal lattice energy is that it can be separated into specific interactions along certain directions and into the constituent atom-atom pairwise contributions. This provides the link between molecular and crystal structures. The calculation of lattice energies thus provides a profile of the important intermolecular interactions that correspond to particular classes of compounds. It also provides an understanding of the nature of the intermolecular interactions that lead to a particular crystal packing arrangement.

An example of a preferred multivariate optimization method used to search for a low energy crystal structure is the hydrogen-bond-biased simulated annealing monte carlo (SAMC) method described by Chin and co-workers in J. Am Chem. Soc. 1999, 121, 2115-2122, the entirety of which is incorporated herein by reference. As described therein, one first builds and parameterizes a molecule using a molecular modeling program such as QUANTA, available from Molecular Simulations Inc., and then minimizes its energy using a program such as CHARMm, also available from Molecular Simulations Inc. (an academic version of the program, referred to as CHARMM, is also available from Harvard University). The molecular frame of reference is preferably positioned at the center of mass of the molecule. Using preset limits of the unit cell and molecular rotation, a trial crystal structure with a given space group is built using a program such as CHARMM. Preferably, the limits used are: (a) a “loose” window for the lengths of the axes of the unit cell (for example, 30% greater than the largest molecular dimension as an upper limit and 3% less than the smallest dimension of the molecule as the lower limit); and (b) a range of angles corresponding to the allowable degree of molecular rotation.

One preferred way of planning additional experiments to find missing expected solid forms is schematically illustrated in FIG. 5: generating a predictive model, such as a regression model, of the experimental parameters and results from the first set of experiments 507, and interpolating or extrapolating those results to determine sets of experimental parameters likely to produce predicted low-energy structures not produced in the first set of experiments 508.

One preferred method for generating a predictive model from the first set of experimental results is to apply Multivariate Adaptive Regression Splines (MARS) to the classified experimental results from the first set of experiments. A computerized implementation of MARS is commercially available from Salford Systems of San Diego, Calif. Other regression methods such as linear regression, stepwise linear regression, additive models (AM), projection pursuit regression (PPR), recursive partitioning regression (RPR), alternating conditional expectations (ACE), additivity and variance stabilization (AVAS), locally weighted regression (LOESS), and neural networks may also be used.

After generating a predictive model, the model can be used to determine a second set of distinct combinations of experimental parameters that, according to the model, should produce predicted solid forms that were not produced in the first set of experiments. This may be accomplished by setting the response variable to a value corresponding to a missing predicted solid form and solving the predictive model for one or more sets of values of experimental parameters giving that result. For preferred predictive models, the solution may be found using algebraic or numerical methods readily apparent to those of ordinary skill in the art of using such predictive models.

Using the process informatics subsystem, the computer-controlled system can be activated to conduct a second set of experiments, each experiment of the second set corresponding to a distinct combination of experimental parameters determined using the predictive model. The second set of experimental results are preferably again compared against predicted experimental results as described above to classify the results according to predicted solid forms and to determine if all predicted low-energy structures have been produced.

Based on the collection of results, an optimum or near-optimum solid form is selected 509. Preferably, data representing the collection of experimental results is processed as a collection of points in a space, such as a topological space, metric space, or vector space comprising dimensions corresponding to the dimensions of the experimental parameters 510. Through such analysis, regions of the space in which the selected solid form is produced, and the boundaries between such regions and regions in which other forms or no solid forms are produced may be determined. Additional sets of experiments may be performed to define such regions with greater resolution 511. Preferably, a set of experimental parameters is thereby determined as far as possible from such boundaries 512. Such a set of parameters is advantageous for manufacture because small variations in manufacturing conditions are less likely to produce a solid form other than the selected form.

VII. Process Informatics and Computational Informatics Subsystems

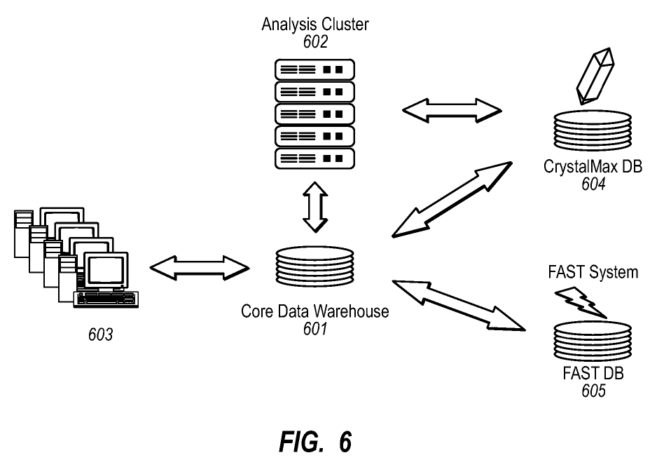

The architecture of one embodiment of a computing system for controlling automated high-throughput systems is schematically illustrated in FIG. 6. The computing system can include a computational informatics subsystem that is comprised of a core data warehouse 601 and an analysis cluster 602. The core data warehouse 601 comprises an Oracle 8i object-oriented relational database management system with partitioning option running under Linux on a Penguin Computing Systems 8500 computer with eight Intel Pentium III 550 megahertz Xeon CPUs and 2 gigabytes of RAM and a one terabyte RAID 5 disk array. The analysis cluster 602 comprises a Penguin Computing Systems Blackfoot dual Intel Pentium III 800 megahertz CPUs with 2 Gigabytes of RAM and 36 gigabytes of disk space running Linux with the MOSIX kernel modification.

The process informatics subsystem comprises a CRYSTALMAX informatics system 604 and a FAST informatics system 605. The CRYSTALMAX informatics system 604 comprises an Oracle 8i object-oriented relational database management system running under Linux on a Penguin Computing Systems 4400 with 4 Intel Pentium Xeon CPUs, 2 gigabytes of RAM and a 500 gigabyte RAID 5 disk array. The FAST informatics system 605 has the same configuration.

Windows systems 603 preferably comprise a variety of personal workstation hardware ranging from typical desktop PCs to high-performance workstations with visualization hardware.

The core data warehouse 601 and analysis cluster 602 are preferably interconnected with gigabit Ethernet. The CRYSTALMAX 604 and FAST 605 informatics systems are also preferably interconnected with the computational informatics subsystem by gigabit Ethernet. Windows systems 603 are typically connected to the computational informatics subsystem by a variety of heterogeneous networks, including the Internet.

However, advances in computer technology can be employed to update the computing system. As such, advanced computer technology can be implemented in the computing system in accordance with the present invention.

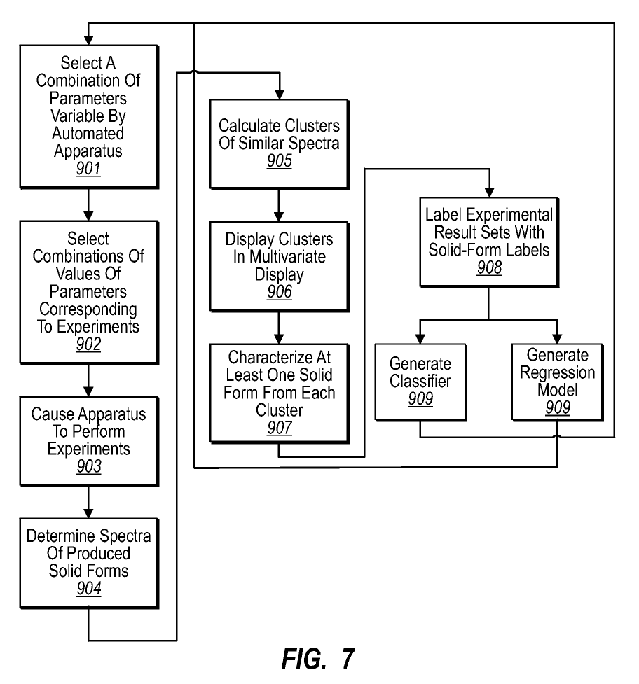

In one embodiment, the computing system can be used in a method to assess a collection of experimental results in a search for novel or known solid forms as schematically illustrated in FIG. 7. The method comprises the steps of: calculating a plurality of clusters of experiments resulting in a solid form based on a measure of similarity of characteristics of the experimental results and/or parameters 905; further characterizing at least one sample solid form from each cluster 907; based on the characterization, assigning a solid form label to each experiment of each cluster 908. The method also comprises additional optional steps of: displaying clusters in a multivariate display 906; generating a classifier to assign a solid form label to an input comprising experimental parameters and/or results 909; generating a regression model 910 to estimate one or more expected property outcomes based on an input comprising experimental parameters and/or results, selecting a combination of experimental parameters variable by an automated experimentation apparatus 901; generating a plurality of sets of values of the experimental parameters, providing one or more of the sets to a classifier and/or regression model as input; based on the output of the classifier and/or regression model, selecting combinations of a plurality of sets of values of experimental parameters corresponding to experiments to be performed 902; providing selected sets of values of experimental parameters to an automated experimentation apparatus 903; and determining Raman spectra for experiments that produce solid forms 904. The method further optionally also comprises providing one or more individual experimental result sets as input to a classifier and/or regression model. The foregoing steps may be iterated an arbitrary number of times, with variations in the steps performed in each iteration. A preferred embodiment for implementing this method comprises the CRYSTALMAX automated experimentation apparatus configured to determine Raman spectra of solid forms, as described more fully in U.S. provisional patent application No. 60/318,138, which is incorporated herein by reference.

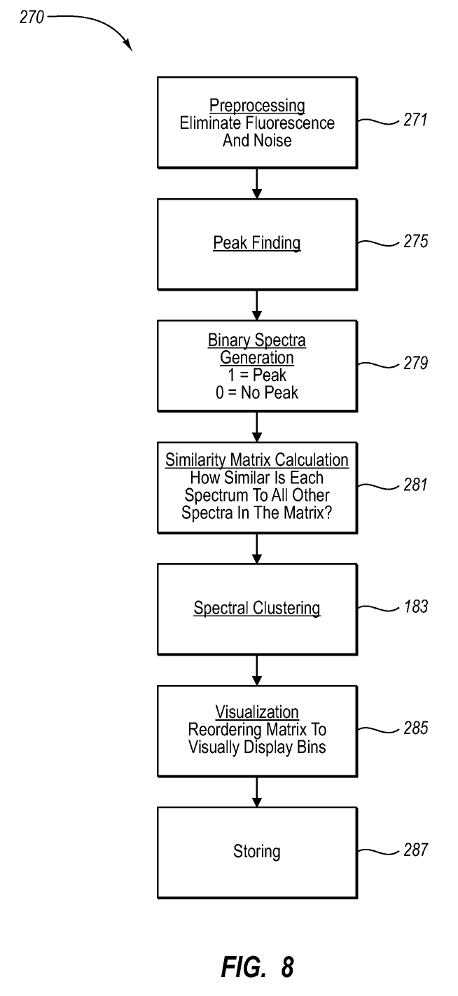

In one preferred embodiment, the computational informatics subsystem receives from the process informatics subsystem a plurality of Raman spectra, each spectrum corresponding to a distinct experiment. The computational informatics subsystem then preferably processes the spectra in six stages as schematically illustrated in the flow chart 270 in FIG. 8: preprocessing 271, peak finding 275, similarity matrix calculation 281, spectral clustering 283, and visualization 285. This process preferably also includes a binary spectra generation stage 279 between peak finding 275 and similarity matrix calculation 281. Each of these stages will be described in detail in the following sections. The following discussion relates to Raman spectra, but the same steps can easily be modified and applied to other types of spectra, or other forms of data.

1. Preprocessing

The purpose of the preprocessing step is to eliminate artifacts of the Raman spectra that are not caused by Raman scattering and to make the Raman scattering peaks as sharp as possible. Raman spectra often contain large fluorescence peaks spread over a broad spectral range and much smaller, narrower peaks caused by measurement, glass background, and instrument noise. Several different filtering techniques can be used in order to eliminate these deleterious features: Fourier filtering, wavelet filtering, matched filtering, and the like. The preferred embodiment uses a matched filter approach where the filter kernel is a zero-mean, symmetric product of sinusoids matched approximately to an average Raman peak width.

Preferably, the bandwidth of the main kernel peak is set to be equal to or slightly smaller than the bandwidth of an average Raman peak. When matched filters of this type are viewed in the Fourier domain, they may be seen to perform as bandpass filters, almost completely attenuating low- and high-frequency spectral components. Furthermore, with the bandwidth of the filter kernel chosen to be equal to or slightly smaller than the average Raman peak bandwidth, this filter detects peaks that are very close to each other. A raw, unfiltered spectrum will often display two close peaks as a main peak with a “shoulder” on one of its sides. After a matched filtering step, though, the shoulder will often be distinguished as a separate peak. This separation is useful for the peak picking procedure described below.

2. Peak Finding

The process of finding peaks in a spectrum is an important aspect of many spectral processing techniques, and there are many commercially available programs for performing this task. Many variations of peak finding algorithms can be found in the literature. An example of a simple algorithm is to find the zero-crossings of the first derivative of a smoothed or unsmoothed spectrum, and then to select the concave down zero-crossings that meet certain height and separation criteria. For the preferred embodiment, the peak finding function available in the software provided with the Almega dispersive Raman spectrometer (Thermo Nicolet, OMNIC software) was used. This function allows the threshold and sensitivity values to be set by the user. The threshold sets the lowest peak height that will be counted as a peak, and the sensitivity controls how far apart each peak must be to count as a separate peak.

3. Binary Spectra Representations

Once the peaks have been found for all of the spectra, binary spectral representations are preferably created for all of the spectra. These binary spectra representations comprise vectors of ones and zeros. Each zero represents the absence of a peak feature and each one represents the presence of a peak feature. A peak feature is simply a peak that occurs within a certain spectral range, preferably a few wave numbers. The vectors for all of the spectra are preferably the same length and corresponding elements of these vectors correspond to the same peak feature.

In order to create these binary spectra, the peaks are clustered into ranges of peak features. The process used to perform this peak clustering is a modified form of a 1-dimensional iterative k-means clustering algorithm. The process begins with the picked peaks from a single spectrum. These peak positions are used to define the centers of peak feature ranges. The peak feature bins cover a range of wave numbers that can be specified by a user (the default is 5 wave numbers). The rest of the spectra are then iteratively added to the peak feature representation. At each step any peak that fits into a pre-existing peak feature range is added to that range. For any peak that does not fit into a range, a new range is created. Centers are not permitted to move so that peak feature ranges overlap. Then, the centers of all of the ranges are re-calculated and the peak feature ranges are re-defined relative to the new centers. This process can leave some peaks outside of an existing peak feature range. In this case, a new range is created for these peaks. This process creates a matrix with each row of the matrix corresponding to a binary spectrum specified in terms of range to which its peaks correspond.

4. Similarity Matrix Calculation

From either the spectra themselves, floating point or integer vectors representing the spectra, or from binary spectra representations such as those generated using the process described above, a similarity measure between pairs of spectra is calculated. Preferably, the similarity measure is calculated between each distinct pair of spectra. This similarity measurement is used to determine one or more clusters of similar spectra. Example similarity measurements include metric distances such as Hamming, Lp, or Euclidean distance, or non-metric similarity indices such as the Tversky similarity index (or its derivatives such as the Tanimoto or Dice coefficients) or functions thereof The selected similarity measure is preferably calculated for each distinct pair of spectra.

5. Spectral Clustering

Using the similarity measure calculated between spectra, a clustering algorithm is applied to determine one or more clusters of similar spectra. A variety of different clustering algorithms may be used.

Hierarchical clustering, including agglomerative and stepwise-optimal hierarchical clustering, k-means clustering, Gaussian mixture model clustering, or self-organizing-map (SOM)-based clustering, clustering using the Chameleon, DBScan, CURE, or Rock clustering algorithms are some of the clustering methods that may be used.

In a preferred embodiment, hierarchical clustering is used as a first-pass method of spectral data processing. Using the information from the hierarchical clustering run, a step of k-means clustering is then performed with user-defined cluster numbers and initial centroid positions.

In another embodiment, the number of clusters can be automatically selected in order to minimize some metric, such as the sum-of-squared error or the trace or determinant of the within cluster scatter matrix.

6. Visualization

Hierarchical clustering produces a dendrogram-sorted list of spectra so that similar spectra are very close to each other. This dendrogram-sorted list is used to rearrange both axes of the original similarity matrix and then present the “sorted similarity” matrix in a coded manner wherein similarity indicia are used for each similarity region, including without limitation different symbols (such as cross-hatching), shades of color, or different colors. In a preferred embodiment, the “sorted similarity” matrix is presented in a color-coded manner, with regions of high similarity in warm colors and regions of low similarity in cool colors. Using this preferred three-dimensional (two spatial dimensions plus color) visualization, many clusters become apparent as warm-colored square regions of similarity along the matrix diagonal. These square regions represent a high degree of similarity between all of the spectral (i,j) pairs in those regions.

It should be noted that the failure of the similarity matrix to present a diagonal form is to be expected with some types of samples, although the matrix is still useful in representing more complex similarity relationships. Furthermore, in some cases there can be similarity regions along more than one possible diagonal that correspond to different rearrangements. Such rearrangements result in off-diagonal similarity square regions becoming part of the diagonal similarity square regions.

Along with the matrix representation of the cluster data, it is also useful to show where all of the spectra and the cluster boundaries lie in a dimensionally reduced space (usually 2-dimensions). There are several ways to perform this dimensionality reduction. In a preferred embodiment, a linear projection is made of a binary spectra matrix onto its first two principal components. Alternatively, the chosen similarity matrix could be used in order to create a map of the data using multidimensional scaling.

An example Raman clustering application is written in Visual Basic (VB). This VB program allows a user to select a group of spectra and set processing parameters. Preprocessing is performed within the VB application and then the filtered spectra are sent to OMNIC for peak finding through the Macros/Pro DDE communication layer provided by OMNIC. Once peaks are found, binary spectrum and distance matrix generation is performed in the main VB application. Then, the distance matrix is sent to MATLAB through a socket communication layer. In MATLAB, clusters are generated and visualizations are created. These visualizations are made available to the main VB application through a web server present on the same machine as the MATLAB instance. The resulting visualization allows for the easy identification of groups of samples that all have similar physical structure.

After clusters have been calculated, it is desirable to correlate clusters with corresponding solid forms. This is preferably accomplished by selecting one sample, or preferably, a plurality of samples from each cluster, and characterizing the selected sample or samples with additional experimental techniques, such as powder X-Ray diffraction and/or differential calorimetry. In a preferred embodiment, the clustering and experimental techniques result in clusters of experimental results all of which produced the same solid form. Based on the additional experimental characterization, solid form labels reflecting the solid form produced by the experiments of the cluster are associated with the experimental result sets by the computational informatics subsystem. These labels are preferably used in combination with the experimental result sets and the corresponding values of experimental parameters to generate one or more regression models and/or classifiers for use in planning and assessing further experiments, or estimating properties for conditions that have not been experimentally verified. For example, regression models may be used to estimate properties over a continuous range reflecting an infinite number of different conditions.

VIII. Data Analysis

In particular embodiments of the invention, spectroscopic data is processed using what is referred to herein as a “spectra binning system,” which allows the rapid analysis and identification of samples in an array by creating, for example, a family or similarity map. Preferred embodiments of the spectra binning system comprise a hardware-based instrumentation platform and a software-based suite of algorithms. The computer software is used to analyze, identify and categorize groups of samples having similar physical forms, thus identifying a group from which the operator, or scientist, can then select a few samples for further analysis. This selection can be performed independently by the scientist, or by using an automated means, such as software designed to automatically select samples of interest. Although, many applications made possible by the spectral binning system will be apparent to those skilled in the art, preferred systems of this invention are used to identify and characterize samples or compounds of interest. Particular binning and analytical methods useful in the invention are disclosed in U.S. patent application Ser. No. 10/142,812, filed May 10, 2002, the entirety of which is incorporated herein by reference.

The spectral binning system is generally used in this invention to detect similarities in the properties of a plurality of samples by observing their binning behavior. Thus, the number of forms of a substance can be estimated by binning spectra. The plurality of samples is examined with a device for generating a corresponding spectrum of acceptable quality (i.e., sufficient S/N ratio). Spectral peaks or other features are next identified to obtain a binary fingerprint. Advantageously, the spectra are compared pairwise in accordance with a metric to generate a similarity score. Other comparisons that use more than two spectra concurrently are also acceptable, although possibly complex.

One or more clustering techniques can be used to generate bins that are preferably well defined, although this is not an absolute requirement since it is acceptable to generate a reduced list of candidate forms for a given substance as an estimate of the heterogeneity of the structure of the substance. Advantageously, the generation of bins facilitates the ready evaluation of structure heterogeneity among samples. For instance, frequency, frequency shift, amplitude, and other similar measurements based on Raman spectra are often limited by the lack of suitable standards. However, the number of bins generated from evaluation of Raman spectra obtained by sampling a substance of interest is a measure that does not directly depend on having a good standard.

The invention also encompasses the use of hierarchical clustering to represent the data in the form of a similarity matrix having similar spectra/samples listed close together. Such a similarity matrix may be sorted to generate similarity regions along a diagonal. The resulting sorted similarity matrix may be used as a basis for setting the number of clusters for k-means clustering or other clustering techniques based on a specified number of clusters such as Gaussian Mixture Modelling.

Advantageously, although the clusters are actually in higher dimensional space, they can be projected into 2 or 3 dimensional space and visualized. Therefore, the binning procedure allows for both steady state and kinetic evaluation of states (e.g. hydration states, crystalline states, and other states or forms that can vary over time). This method is well-suited for such measurements since individual Raman spectra can be collected rapidly (e.g. in a few seconds). Preferably, the turn-around time for generating a spectrum and assigning the spectrum to a bin is less than about two minutes, one minute, ten seconds, or one second. Moreover, limited real time processing is often possible if an acquired spectrum is to be assigned to existing bins; or, in a preferred embodiment of the invention, a library of binned spectra is updated with newly acquired spectra. In a preferred embodiment, newly acquired spectra from a single sample may all be binned into a single bin based on a majority of them being more related to the single bin in accordance with a metric, such as those discussed below and elsewhere herein.

Once the spectra from all of the samples to be analyzed have been collected, they are processed by a series of algorithms. These algorithms facilitate the binning of sample spectra according to one or more spectral features. Examples of such features include, but are not limited to, the locations of peaks, peak shoulders, peak heights, and peak areas. In a preferred embodiment, the spectral binning process bins spectra based on the locations of their scattering peaks and peak shoulders, expressed as wavelength or Raman shift (cm⁻¹).

In the spectra binning system, the collected spectra can be binned using the raw or filtered spectra, peak height spectra generated using peaks selected from the raw or filtered spectra, and binary spectra generated using the raw or filtered spectra.

IX. Maximally Diverse Values of Experimental Parameters

One preferred approach to generating the first set of experiments in what may be a succession of iterative experiments is to systematically create a diverse set of experiments in a property/descriptor space of potential interest. Experimental parameters that may be varied by the automated experimentation apparatus must be selected, and values for those parameters determined, in order to conduct a set of experiments. Parameters may be selected by scientists acting on knowledge of the chemistry of the compound of interest, or the computational informatics system may guide the selection or suggest parameters by querying the database for similar compounds of interest and analyzing which descriptors were significant in prior experiments and/or simulations. The descriptors may then be mapped onto parameters that may be varied by the automated experimentation apparatus.

Many methods for solving the parameter selection problem in QSAR/QSPR are known. Three of the most popular solutions involve stepwise algorithms, genetic algorithms, and simulated annealing. These approaches may be adapted to parameter selection in the present computer-controlled system.

Stepwise algorithms are straightforward, but can lead to suboptimal results. A regression or classification is performed using each possible independent variable. The variable that performs the best is added to the model. The regression or classification is then performed again with the first variable and all possible second variables. The best second variable is then added to the model. Additional variables are added in similar fashion. This process is preferably continued a set number of times or until some measure of predictive ability reaches a minimum.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. In a computing system designed for controlling automated high-throughput processing of an array having a large number of samples in order to identify chemical and/or physical properties leading to optimal formulation for a given use of a compound of interest, a method of computer-aided design for determining an experimental formulation for each sample, each experimental formulation being based on at least one experimental variable which is varied as to at least some samples so that the effect in terms of changes in the chemical and/or physical properties of the compound of interest due to at least one experimental variable can be identified across a large number of comparative samples for a compound of interest, the method comprising:
inputting into the computing system at least one compound of interest to be included in each of a plurality of experimental formulations that are to be designed for the array of samples;
inputting into the computer system additional components to be formulated with the at least one compound of interest in the experimental formulations;
inputting into the computing system at least one experimental variable to be varied as between at least some of the samples of the array; and
the computing system thereafter designing a plurality of unique experimental formulations that differ as between at least some samples of the array based on at least one experimental variable that is varied as between the at least some samples of the array, each experimental formulation being designed at least in part based on at least one experimental variable.

2. In a computing system designed for controlling automated high-throughput processing of an array having a large number of samples in order to identify chemical and/or physical properties leading to optimal formulation for a given use of a compound of interest, a computer-program product for implementing a method of computer-aided design for determining an experimental formulation for each sample, each experimental formulation being based on at least one experimental variable which is varied as to at least some samples so that the effect in terms of changes in the chemical and/or physical properties of the compound of interest due to at least one experimental variable can be identified across a large number of comparative samples for a compound of interest, the computer-program product comprising a computer-readable medium containing computer-executable instructions for causing the computing system to execute the method, and wherein the method is comprised of:
inputting into the computing system at least one compound of interest and any additional components to be included in each of a plurality of experimental formulations that are to be designed for the array of samples;
inputting into the computer system additional components to be formulated with the at least one compound of interest in the experimental formulations;
inputting into the computing system at least one experimental variable to be varied as between at least some of the samples of the array; and
the computing system thereafter designing a plurality of unique experimental formulations that differ as between at least some samples based on at least one experimental variable that is varied as between the at least some samples of the array, each experimental formulation being designed at least in part based on at least one experimental variable.

3. A method as in claims 1 or 2 wherein the at least one experimental variable to be varied as between at least some samples of the array is varied as to at least one of the following:
concentration of the compound of interest, concentration of components in the experimental formulations, identity of the components, combination of components, additive, solvent, antisolvent composition, temperature, temperature change, heating, cooling, nucleation seeds, supersaturation, pH, pH change, or time of crystallization reaction.

4. A method as in claims 1 or 2, further comprising inputting into the computing system at least one criteria for determining the effect of at least one experimental variable for each experimental formulation that is varied as to that experimental variable, wherein said effect is manifested by a change in one or more of the following for a compound of interest between different experimental formulations:
microstructure, crystallinity, amorphism, polymorphism, hydrate, solvate, isomorphic desolvate, packing order, ionic crystal, interstitial space, lattice, or habit.

5. A method as in claims 1 or 2, further comprising the computing system designing a process for processing the array of samples to determine an effect on the compound of interest of at least one experimental variable for each experimental formulation.

6. A method as in claim 5, wherein the processing of each experimental formulation includes a process consisting of at least one of the following:
mixing, agitating, heating, cooling, adjusting pressure, adding crystallization aids, adding nucleation promoters, adding nucleation inhibitors, adding acids, adding bases, stirring, milling, filtering, centrifuging, emulsifying, mechanically stimulating, introducing ultrasound energy to the experimental formulation, introducing laser energy to the experimental formulation, subjecting the experimental formulation to a temperature gradient, allowing the experimental formulation to set for a time, or heating to a first temperature then cooling to a second temperature.

7. A method as in claim 5, wherein the effect is at least one of causing crystallization, inhibiting crystallization, or formation of a solid form.

8. In a computing system designed for controlling automated high-throughput processing of an array having a large number of samples in order to identify chemical and/or physical properties leading to optimal formulation for a given use of a compound of interest represented in the array, a method of computer-aided design for determining an experimental formulation for each sample, each experimental formulation being based on at least one experimental variable which is varied as to at least some samples so that the effect in terms of changes in the chemical and/or physical properties of the compound of interest due to at least one experimental variable can be identified across a large number of comparative samples for the compound of interest, the method comprising:
inputting into the computing system a compound of interest to be included in each of a plurality of experimental formulations that are to be designed for the array of samples;
inputting into the computer system a plurality of additional components to be formulated with the compound of interest in the experimental formulations;
inputting into the computing system a plurality of experimental variables to be varied as between at least some of the samples of the array;
the computing system thereafter designing, for a first group of samples in the array, a first plurality of experimental formulations that are different as between at least some of the samples in the first group that are based on a first experimental variable that is varied among the first plurality of experimental formulations determined for the first group; and
the computing system also designing, for at least a second group of samples in the array, a second plurality of experimental formulations that are different as between at least some of the samples in the second group that are based on a second experimental variable that is varied as among the second plurality of experimental formulations determined for the second group.

9. In a computing system designed for controlling automated high-throughput processing of an array having a large number of samples in order to identify chemical and/or physical properties leading to optimal formulation for a given use of a compound of interest represented in the array, a computer-program product for implementing a method of computer-aided design for determining an experimental formulation for each sample, each experimental formulation being based on at least one experimental variable which is varied as to at least some samples so that the effect in terms of changes in the chemical and/or physical properties of the compound of interest due to at least one experimental variable can be identified across a large number of comparative samples for the compound of interest, the computer-program product comprising a computer-readable medium for containing computer-executable instructions for causing the computing system to execute the method, and wherein the method is comprised of:
inputting into the computing system a compound of interest to be included in each of a plurality of experimental formulations that are to be designed for the array of samples;
inputting into the computer system a plurality of additional components to be formulated with the compound of interest in the experimental formulations;
inputting into the computing system a plurality of experimental variables to be varied as between at least some of the samples of the array;
the computing system thereafter designing, for a first group of samples in the array a first plurality of experimental formulations that are different as between at least some of the samples in the first group that are based on a first experimental variable that is varied among the first plurality of experimental formulations determined for the first group; and
the computing system also designing, for at least a second group of samples in the array a second plurality of experimental formulations that are different as between at least some of the samples in the second group that are based on a second experimental variable that is varied as among the second plurality of experimental formulations determined for the second group.

10. A method as in claims 8 or 9, wherein the plurality of experimental variables to be varied as between at least some of the samples of the array include at least one of the following:
concentration of the compound of interest, concentration of components in the experimental formulations, identity of components, combination of components, additive, solvent, antisolvent composition, temperature, temperature change, heating, cooling, nucleation seeds, supersaturation, pH, pH change, or time of crystallization reaction;

11. A method as in claims 8 or 9, further comprising inputting into the computing system at least one criteria for determining the effect of at least one experimental variable for each experimental formulation that is varied as to that experimental variable, wherein said effect is manifested by a change in one or more of the following for a compound of interest between different experimental formulations:
microstructure, crystallinity, amorphism, polymorphism, hydrate, solvate, isomorphic desolvate, packing order, ionic crystal, interstitial space, lattice, or habit.

12. A method as in claims 8 or 9, further comprising the computing system designing a process for processing each of the experimental formulations in the array of samples to determine an effect on a compound of interest of at least one experimental variable for each experimental formulation.

13. A method as in claim 12, wherein the processing of each experimental formulation includes a process consisting of at least one of the following:
mixing, agitating, heating, cooling, adjusting pressure, adding crystallization aids, adding nucleation promoters, adding nucleation inhibitors, adding acids, adding bases, stirring, milling, filtering, centrifuging, emulsifying, mechanical stimulation, introducing ultrasound energy to the experimental formulation, introducing laser energy to the experimental formulation, subjecting the experimental formulation to a temperature gradient, allowing the experimental formulation to set for a time, or heating to a first temperature then cooling to a second temperature.

14. A method as in claim 12, wherein the effect is at least one of causing crystallization, inhibiting crystallization, or formation of a solid form.

15. In a computing system designed for controlling automated high-throughput processing of an array having a large number of samples in order to identify chemical and/or physical properties leading to optimal formulation for a given use of a compound of interest, and wherein the computing system provides computer-aided design and processing of an experimental formulation for each sample, each experimental formulation having the compound of interest and being based on at least one experimental variable which is varied as to at least some samples so that the effect in terms of changes in the chemical and/or physical properties of the compound of interest due to at least one experimental variable can be identified across a large number of comparative samples, a method of analyzing data from the large number of comparative samples comprising:
inputting into the computing system at least one compound of interest and any additional components to be included in a plurality of experimental formulations that are to be designed for the array of samples;
inputting into the computing system at least one selected experimental variable of interest that is to be varied as between at least some of the samples of the array;
the computing system thereafter designing a plurality of unique experimental formulations that differ as between at least some samples of the array based on the at least one selected experimental variable of interest that is varied as between the at least some samples of the array;
the computing system thereafter controlling a process by which an experimental formulation for each sample is prepared and tested in order to create changes across a large number of comparative samples for the at least one compound of interest in its chemical and/or physical properties;
inputting into the computing system detected changes across the large number of comparative samples for the at least one compound of interest; and
the computing system thereafter automatically screening the large number of samples by identifying those samples which contain chemical and/or physical properties likely to lead to an optimal formulation for a given use of a compound of interest.

16. In a computing system designed for controlling automated high-throughput processing of an array having a large number of samples in order to identify chemical and/or physical properties leading to optimal formulation for a given use of a compound of interest, and wherein the computing system provides computer-aided design and processing of an experimental formulation for each sample, each experimental formulation having the compound of interest and being based on at least one experimental variable which is varied as to at least some samples so that the effect in terms of changes in the chemical and/or physical properties of the compound of interest due to at least one experimental variable can be identified across a large number of comparative samples, a computer-program product for implementing a method of analyzing data from the large number of comparative samples, the computer-program product comprising a computer-readable medium containing computer-executable instructions for causing the computing system to execute the method, and wherein the method is comprised of:
inputting into the computing system at least one compound of interest and any additional components to be included in a plurality of experimental formulations that are to be designed for the array of samples;
inputting into the computing system at least one selected experimental variable of interest that is to be varied as between at least some of the samples of the array;
the computing system thereafter designing a plurality of unique experimental formulations that differ as between at least some samples of the array based on the at least one selected experimental variable of interest that is varied as between the at least some samples of the array;
the computing system thereafter controlling a process by which an experimental formulation for each sample is prepared and tested in order to create changes across a large number of comparative samples for the at least one compound of interest in its chemical and/or physical properties;
inputting into the computing system detected changes across the large number of comparative samples for the at least one compound of interest; and
the computing system thereafter automatically screening the large number of samples by identifying those samples which contain chemical and/or physical properties likely to lead to an optimal formulation for a given use of a compound of interest.

17. A method as in claims 15 or 16, wherein the at least one selected experimental variable of interest that is to be varied as between at least some samples of the array is varied as to at least one of the following:
concentrations of the compound of interest, concentrations of components in the experimental formulations, identity of components, combination of components, additives, solvents, antisolvent compositions, temperatures, temperature changes, heating, cooling, nucleation seeds, supersaturation, pH, pH change, or time of crystallization reaction.

18. A method as in claims 15 or 16, the chemical and/or physical properties likely to lead to optimal formulation for a given use of a compound of interest being at least one of microstructure, crystallinity, amorphism, polymorphism, hydrate, solvate, isomorphic desolvate, packing order, ionic crystal, interstitial space, lattice, or habit.

19. A method as in claims 15 or 16, further comprising:
inputting into the computing system a data set, based on analyzing the preparation and processing of each of the experimental formulations in the array of sample, having experimental data for the changes across the large number of comparative samples for the at least one compound of interest; and
analyzing the data set to determine at least one optimal formulation for a given use of a compound of interest.

20. A method as in claims 15 or 16, wherein the computing system further determines a process for processing each of the experimental formulations in the array of samples.

21. A method as in claim 20, wherein the processing of each experimental formulation includes a process consisting of at least one of the following:
mixing, agitating, heating, cooling, adjusting pressure, adding crystallization aids, adding nucleation promoters, adding nucleation inhibitors, adding acids, adding bases, stirring, milling, filtering, centrifuging, emulsifying, mechanical stimulation, introducing ultrasound energy to the experimental formulation, introducing laser energy to the experimental formulation, subjecting the experimental formulation to a temperature gradient, allowing the experimental formulation to set for a time, or heating to a first temperature then cooling to a second temperature.

22. A method as in claims 15 or 16, wherein the effect in terms of changes in the chemical and/or physical properties of the compound of interest due to at least one experimental variable causes at least one of crystallization, inhibiting crystallization, or formation of a solid form.

23. A method as in claims 15 or 16, further comprising:
inputting into the computer system information obtained by screening the chemical and/or physical properties of each of the experimental formulations in the array of samples for at least one desired property; and
the computing system identifying at least one experimental formulation having the at least one desired property based on the obtained information.

24. In a computing system designed for controlling automated high-throughput processing of an array having a large number of samples in order to identify chemical and/or physical properties leading to optimal formulation for a given use of a compound of interest, and wherein the computing system provides computer-aided design and processing of an experimental formulation for each sample, each experimental formulation having the compound of interest and being based on at least one experimental variable which is varied as to at least some samples so that the effect in terms of changes in the chemical and/or physical properties of the compound of interest due to at least one experimental variable can be identified across a large number of comparative samples, a method of analyzing data from the large number of comparative samples comprising:
inputting into the computing system at least one compound of interest and any additional components to be included in a plurality of experimental formulations that are to be designed for a first array of samples;
inputting into the computing system at least one selected experimental variable of interest that is to be varied as between at least some samples of the first array;
the computing system thereafter designing a plurality of unique experimental formulations that differ as between at least some samples based on the at least one selected experimental variable of interest that is varied as between the at least some samples of the first array;
the computing system thereafter controlling a process by which an experimental formulation for each sample is prepared and tested in order to create changes in chemical and/or physical properties across a large number of comparative samples for the at least one compound of interest;
inputting into the computing system detected changes across the large number of comparative samples for the at least one compound of interest;
the computing system thereafter screening the large number of samples by identifying those samples which contain chemical and/or physical properties likely to lead to an optimal formulation for a given use of a compound of interest, and storing as a first data set information as to the experimental formulation and the resulting chemical and/or physical properties for each of the identified samples;
inputting to the computing system at least one other selected experimental variable of interest that is to be varied as between at least some identified samples of the first data set;
the computing system thereafter designing a plurality of further experimental formulations for a second array having a large number of samples that are different as between at least some of the identified samples of the first data set based on the at least one further selected experimental variable of interest that is to be varied as between the at least some identified samples of the first data set;
the computing system thereafter controlling a process by which the plurality of further experimental formulations in the second array of samples are prepared and tested in order to create further changes in chemical and/or physical properties across further comparative samples for the at least one compound of interest;
inputting into the computing system detected further changes across the further comparative samples of the first data set for the at least one compound of interest;
the computing system thereafter screening the further comparative samples by identifying changes in chemical and/or physical properties and storing as a second data set information as to the plurality of further experimental formulations and the resulting chemical and/or physical properties for each further comparative sample; and
the computing system thereafter selecting from the first and second data sets those samples which contain chemical and/or physical properties likely to lead to an optimal formulation for a given use of a compound of interest.

25. In a computing system designed for controlling automated high-throughput processing of an array having a large number of samples in order to identify chemical and/or physical properties leading to optimal formulation for a given use of a compound of interest, and wherein the computing system provides computer-aided design and processing of an experimental formulation for each sample, each experimental formulation being based on at least one experimental variable which is varied as to at least some samples so that the effect in terms of changes in the chemical and/or physical properties of the compound of interest due to at least one experimental variable can be identified across a large number of comparative samples for a compound of interest, a computer-program product for implementing a method of analyzing data from the large number of comparative samples, the computer-program product comprising a computer-readable medium containing computer-executable instructions for causing the computing system to execute the method, and wherein the method is comprised of:
inputting into the computing system at least one compound of interest and any additional components to be included in each of a plurality of experimental formulations that are to be designed for the array of samples;
inputting into the computing system at least one selected experimental variable of interest that is to be varied as between at least some samples of the array;
the computing system thereafter designing a plurality of unique experimental formulations that differ as between at least some samples based on the at least one selected experimental variable of interest that is varied as between the at least some samples of the array;
the computing system thereafter controlling a process by which an experimental formulation for each sample is tested in order to create changes in chemical and/or physical properties across a large number of comparative samples for the at least one compound of interest;
inputting into the computing system detected changes across the large number of comparative samples for the at least one compound of interest;
the computing system thereafter screening the large number of samples by identifying those samples which contain chemical and/or physical properties likely to lead to an optimal formulation for a given use of a compound of interest, and storing as a first data set information as to the experimental formulation and the resulting chemical and/or physical properties for each of the identified samples;
inputting to the computing system at least one other selected experimental variable of interest that is to be varied as between at least some identified samples of the first data set;
the computing system thereafter designing a plurality of further experimental formulations that for a second array having a large number of samples that are different as between at least some of the identified samples of the first data set based on the at least one further selected experimental variable of interest that is to be varied as between the at least some identified samples of the first data set;
the computing system thereafter controlling a process by which the plurality of further experimental formulations in the second array of samples are prepared and tested in order to create further changes in chemical and/or physical properties across further comparative samples for the at least one compound of interest;
inputting into the computing system detected further changes across the further comparative samples of the first data set for the at least one compound of interest;
the computing system thereafter screening the further comparative samples of the first data set by identifying changes in chemical and/or physical properties and storing as a second data set information as to the plurality of further experimental formulations and the resulting chemical and/or physical properties for each further comparative sample; and
the computing system thereafter selecting from the first and second data sets those samples which contain chemical and/or physical properties likely to lead to an optimal formulation for a given use of a compound of interest.

26. A method as in claims 24 or 25, wherein the at least one selected experimental variable of interest and the at least one further experimental variable interest that are to be varied as between at least some samples of the array are each varied as to at least one of the following:
concentration of the compound of interest, concentration of components in the experimental formulations, identity of components, combination of components, additive, solvent, antisolvent composition, temperature, temperature change, heating, cooling, nucleation seeds, supersaturation, pH, pH change, or time of crystallization reaction.

27. A method as in claims 24 or 25, the chemical and/or physical properties likely to lead to optimal formulation for a given use of a compound of interest being at least one of microstructure, crystallinity, amorphism, polymorphism, hydrate, solvate, isomorphic desolvate, packing order, ionic crystal, interstitial space, lattice, or habit.

28. A method as in claims 24 or 25, further comprising:
inputting into the computing system a data set, based on analyzing the preparation and processing of each of the experimental formulations in the array of sample, having experimental data for the changes across the large number of comparative samples or further comparative samples for the at least one compound of interest; and
analyzing the data set to determine at least one optimal formulation for a given use of a compound of interest.

29. A method as in claims 24 or 25, the computing system further determining a process for processing each of the experimental formulations in the first or second array of samples.

30. A method as in claim 29, wherein the processing of each experimental formulation includes a process consisting of at least one of the following:
mixing, agitating, heating, cooling, adjusting pressure, adding crystallization aids, adding nucleation promoters, adding nucleation inhibitors, adding acids, adding bases, stirring, milling, filtering, centrifuging, emulsifying, mechanical stimulation, introducing ultrasound energy to the experimental formulation, introducing laser energy to the experimental formulation, subjecting the experimental formulation to a temperature gradient, allowing the experimental formulation to set for a time, or heating to a first temperature then cooling to a second temperature.

31. A method as in claims 24 or 25, wherein the effect in terms of changes in the chemical and/or physical properties of the compound of interest due to at least one experimental variable causes at least one of crystallization, inhibiting crystallization, or formation of a solid form.

32. A method as in claims 24 or 25, further comprising:
the computer system at least partially controlling or assisting in screening the chemical and/or physical properties of each of the experimental formulations in the array of samples for at least one desired property; and
the computer system at least partially controlling or assisting in identifying at least one experimental formulation having the at least one desired property.

33. A method as in claims 24 or 25, wherein each experimental formulation in the first array of samples has a different combination of any additional components.

34. A method as in claim 33, wherein a first set of the plurality of further experimental formulations in the second array of samples has a different concentration of at least one additional component in at least one experimental formulation of the first array of samples.

35. A method as in claims 24 or 25, wherein the at least one selected experimental variable of interest includes identity of any additional components.

36. A method as in claim 35, wherein the at least one further selected experimental variable of interest includes a concentration gradient for at least one selected additional component.

37. A method as in claim 35, wherein the at least one further selected experimental variable of interest includes a concentration gradient for the at least one compound of interest.
